(19) United States
(12) Patent Application Publication
(10) Pub. No.: US 2011/0319629 A1
(43) Pub. Date: Dec. 29, 2011

(54) TITANIUM COMPOUNDS AND PROCESS FOR CYANATION OF IMINES

(75) Inventors: Abdul Majeed Seayad, Parc Oasis (SG); Balamurugam Ramalingam, Singapore (SG); Christina Chai, Jurong Island (SG); Takushi Nagata, Jurong Island (SG); Kazuhiko Yoshinaga, Jurong Island (SG); Manabu Wadamoto, Singapore (SG)

(73) Assignees: Agency for Science, Technology and Research, Connexis (SG); Mitsui Chemicals Inc., Tokyo (JP)

(21) Appl. No.: 13/201,053

(22) PCT Filed: Feb. 11, 2010
(86) PCT No.: PCT/SG2010/000052

§ 371 Date: Aug. 11, 2011

(30) Foreign Application Priority Data
Feb. 11, 2009 (SG) PCT/SG2009/000044

(51) Int. Cl. C07D 213/57; C07D 333/50; C07D 307/02; C07F 7/28; C07C 253/00

(52) U.S. Cl. 546/330; 556/56; 558/315; 549/492; 549/49

(57) ABSTRACT

The present invention relates to titanium catalysts for synthesis reactions produced by bringing a reaction mixture comprising a titanium alkoxide and a ligand in contact with water, wherein the ligand is represented by the general formula (e): wherein R¹, R², R³, and R⁴ are independently a hydrogen atom, an alkyl group, or the like, and (A) represents a group with two or more carbon atoms. The titanium catalysts may be isolated in solid form and may be stored. The invention further relates to a process for cyanation of imines, wherein the process comprises reacting an imine with a cyanating agent in the presence of the titanium catalyst.

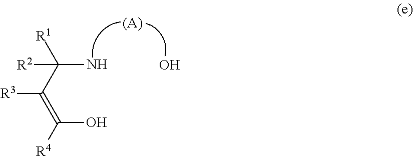

### FIELD OF THE INVENTION

The present invention relates to a titanium compound and a process for producing optically active alpha-aminonitriles according to the asymmetric cyanation reaction of an imine using such a titanium compound. The optically active alpha-aminonitriles are useful as intermediates in the synthesis of pharmaceuticals and fine-chemicals.

### BACKGROUND OF THE INVENTION

One of the oldest, most efficient and economic methods of synthesizing alpha-amino acids is the use of a three component Strecker reaction of aldehydes or ketones with ammonia (or an equivalent) in the presence of a cyanide source. Subsequent hydrolysis of the resultant aminonitrile yields the corresponding alpha-amino acids, as shown by the reaction in FIG. 1A. FIG. 1B shows a modified Strecker reaction, a popular and widely used alternative route for synthesizing alpha-amino acids, wherein an amine is used instead of ammonia and pre-formation of imines is followed by hydrocyanation.

Despite the efficiency and versatility of the Strecker reaction, no catalytic asymmetric version of the reaction or catalytic asymmetric hydrocyanation of imines was reported until the mid-1990s. Since then there has been considerable advances in the development of efficient asymmetric processes for the synthesis of optically active alpha-amino acids, especially nonproteinogenic alpha-amino acids. Both organometallic- and organo-catalysts have also been used in the asymmetric hydrocyanation of imines to produce the corresponding chiral alpha-aminonitriles in the presence of a suitable cyanide source. Although good to excellent results have been reported, many of these catalyst systems utilize expensive ligands and catalysts that are prepared through multi-step synthesis, as well as rigorous conditions such as low temperatures.

Accordingly, improved compound and methods for asymmetric hydrocyanation of imines are needed.

### SUMMARY OF THE INVENTION

In some embodiments, the present invention provides titanium catalysts, produced by bringing water into contact with a complex prepared from a titanium alkoxide represented by the general formula (d),

Ti(OR′)ₓY₍₄₋ₓ₎ (d),

wherein R′ can be the same or different and is an alkyl, an alkenyl, or an aryl group, optionally substituted; Y can be the same or different and in a halogen atom, an acyl group, or an acetylacetonate group; and x is an integer having a value between 0 and 4; and a ligand represented by the formula (e),

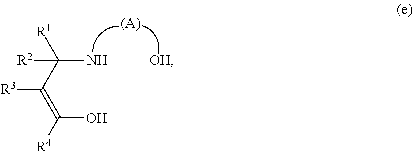

wherein R¹, R², R³, and R⁴ are independently a hydrogen atom, an alkyl group, an alkenyl group, an aryl group, an aromatic heterocyclic group, a non-aromatic heterocyclic group, an acyl group, a hydroxyl group, an alkoxy group, an aryloxy group, an amino group, a cyano group, a nitro group, a silyl group, a siloxy group, an alkoxycarbonyl group or an aryloxycarbonyl group, each of which may have a substituent, or two or more of R¹, R², R³, and R⁴ may be linked together to form a ring, and the ring may have a substituent; and (A) represents a group with two or more carbon atoms.

In some cases, the present invention provides isolated titanium catalysts for synthesis reactions, comprising a compound represented by the general formula (f),

[Tiₘ(L)ₙ(OR′)ₚ(O)_{q}(OH)ᵣYₛ] (f)

wherein R′ can be the same or different and is an alkyl, an alkenyl, or an aryl group, optionally substituted; Y can be the same or different and is a halogen atom, an acyl group, or an acetylacetonate group; m is an integer greater than 1; n and q are the same or different and are 0 or integers greater than 0; p, r, and s are the same or different and are 0 or an integer greater than 0; and L is a ligand represented by the general formula (e),

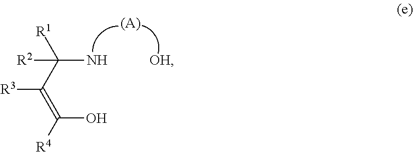

wherein R¹, R², R³, and R⁴ are independently a hydrogen atom, an alkyl group, an alkenyl group, an aryl group, an aromatic heterocyclic group, a non-aromatic heterocyclic group, an acyl group, a hydroxyl group, an alkoxy group, an aryloxy group, an amino group, a cyano group, a nitro group, a silyl group, a siloxy group, an alkoxycarbonyl group or an aryloxycarbonyl group, each of which may have a substituent, or two or more of R¹, R², R³, and R⁴ may be linked together to form a ring, and the ring may have a substituent; and (A) represents a group with two or more carbon atoms.

In some embodiments, the isolated titanium catalyst comprises a compound as represented by any one of the following formulas:

[Tiₘ(L)ₙ(OH)ᵣ] (g),

[Tiₘ(L)ₙ(O)_{q}(OH)ᵣ] (h), or

[Tiₘ(L)ₙ(OR′)ₚ(OH)ᵣ] (i).

In some embodiments, the isolated titanium catalyst comprises a compound as represented by any one of the following formulas:

[Ti₃(L)₃(OH)₆] (j),

[Ti₃(L)₃(O)₃(OH)₃] (k), or

[Ti₃(L)₂(OR′)₃(OH)₃] (l).

The present invention also provides processes for asymmetric cyanation of imines, comprising reacting an imine with a cyanating agent in the presence of a titanium catalyst of the invention. In some embodiments, the imine is represented by the general formula (c),

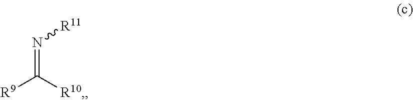

wherein R⁹ and R¹⁰ are independently a hydrogen atom, an alkyl group, an alkenyl group, an alkynyl group, an aryl group, an aromatic heterocyclic group or a non-aromatic heterocyclic group, each of which may have a substituent, and R⁹ is different from R¹⁰; R⁹ and R¹⁰ may be linked together to form a ring, and the ring may have a substituent; R¹¹ is a hydrogen atom, an alkyl group, an alkenyl group, an alkynyl group, an aryl group, an aromatic heterocyclic group or a non-aromatic heterocyclic group, a phosphonate, phosphinoyl, phosphine oxide, alkoxycarbonyl, sulfinyl, or sulfoxy group, each of which may have a substituent; and R¹¹ may be linked either to R⁹ or R¹⁰ to form a ring through a carbon chain, and the ring may have substituents.

Processes for asymmetric cyanation of imines may comprise reacting an imine and a cyanating agent in the presence of a catalyst to form an optically active alpha-aminonitrile, wherein the catalyst is present in an amount from about 0.5 to 30 mol %, relative to the imine. In some embodiments, the catalyst is present in an amount less than 10 mol % (e.g., from 2.5 to 5.0 mol %), relative to the imine. The process may be conducted at any temperature and with any reaction time suited for a particular application. In some embodiments, the process is conducted at a reaction temperature between −78° C. and 80° C. In some embodiments, the process may comprise reacting an imine and a cyanating agent in the presence of a catalyst at a room temperature and/or with a reaction time of less than six hours, or less than two hours, and with a high yield, and wherein the optically active alpha-aminonitrile is obtained in good to excellent enantiomeric excess (e.g., at least 90%).

### BRIEF DESCRIPTION OF THE DRAWINGS

FIG. 1A shows the synthesis of an alpha-amino acid via a Strecker reaction and subsequent hydrolysis of the resultant aminonitrile.

FIG. 1B shows the synthesis of an alpha-amino acid modified Strecker reaction and subsequent hydrolysis of the resultant aminonitrile.

FIG. 2A shows the synthesis of a titanium alkoxide-ligand precatalyst.

FIGS. 2B and 2C show non-limiting examples of the synthesis of a titanium catalyst via a titanium alkoxide-ligand precatalyst.

FIG. 3A shows an asymmetric cyanation of a benzhydryl imine in the presence of an optically active titanium catalyst of the invention, trimethylsilyl cyanide, and n-butanol, according to one embodiment of the invention.

FIG. 3B shows an asymmetric cyanation of a benzyl imine in the presence of an optically active titanium catalyst of the invention, trimethylsilyl cyanide, and n-butanol, according to one embodiment of the invention.

FIGS. 4A and 4B show mass spectroscopy data and some non-limiting examples of plausible structural fragments, according to some embodiments.

FIG. 5 shows a graph of thermogravimetric data for SCTC-Bn material obtained from Example 4.

FIG. 6 shows a graph of thermogravimetric data for SCTC-iPr material obtained from Example 5.

FIG. 7 shows a graph of thermogravimetric data for SCTC-t-Bu material obtained from Example 6.

FIG. 8 shows the IR spectra for a Bn-ligand (top) and SCTC-Bn material obtained from Example 4 (top)

FIG. 9 shows the IR spectra for a iPr-ligand (top) and SCTC-iPr material obtained from Example 5 (bottom).

FIG. 10 shows the IR spectra for a t-Bu-ligand (top) and SCTC-t-Bu material obtained from Example 6 (bottom).

FIG. 11 shows the IR spectra for a Ind-ligand (top) and SCTC-Ind material obtained from Example 85 (bottom).

FIG. 12 shows an SEM image of the SCTC-Bn material obtained from Example 4.

FIG. 13 shows an SEM image of the SCTC-Ind Material obtained from Example 85.

FIG. 14 shows graphs of the XPS data for SCTC material isolated from Example 4.

FIG. 15. shows examples of various coordination states of a ligand, according to one embodiment.

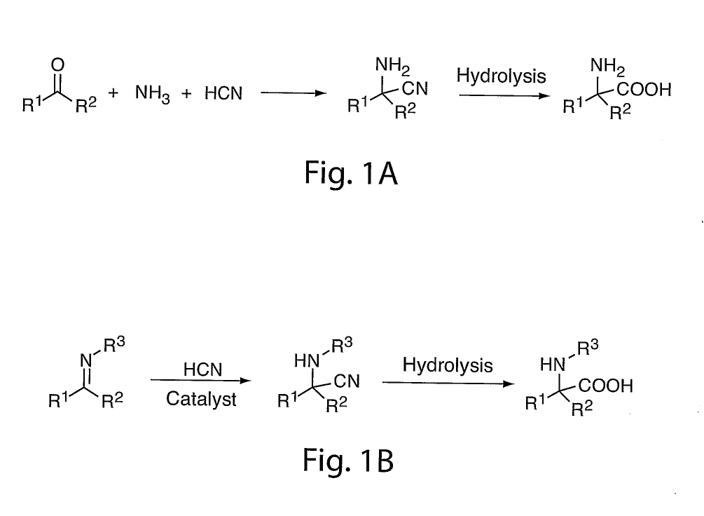

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention relates to a titanium compound and a process for producing alpha-aminonitriles according to the cyanation reaction of an imine using such a titanium compound. Some embodiments of the present invention relate to a titanium compound and a process for producing optically-active alpha-aminonitriles according to the asymmetric cyanation reaction of an imine using such a titanium compound.

Compounds (e.g., catalysts) and methods of the invention involve titanium catalysts useful for synthesis reactions (e.g., asymmetric synthesis reactions), including carbon-carbon bond forming reactions. In some embodiments, the present invention provides catalysts and related methods for asymmetric Strecker-type reactions, such as the asymmetric cyanation of imines for the synthesis of optically active alpha-aminonitriles. The present invention provides efficient catalysts based on inexpensive, stable ligands derived from readily available building blocks. Catalysts and methods of the invention that may advantageously be used under mild reaction conditions, such as room temperature and/or under ambient conditions, to achieve high yields (e.g., >90%, >95%, >98%, >99%) and excellent enantioselectivities (e.g., >90%, >95%, >98%).

In some embodiments, the catalysts may also be isolated (e.g., as a solid). For example, the catalyst may be isolated as a solid chiral titanium compound. In some cases, solid catalysts may also exhibit improved stability relative to, for example, an essentially identical catalyst that is partially hydrolyzed. In some cases, the catalyst may be stored for extended periods of time (e.g., >1 day, >1 month, >6 months, >1 year, etc.). In some embodiments, a solid catalyst form may have improved stability to air and moisture relative.

In some cases, the catalysts described herein may advantageously be recycled, i.e., recovered upon reaction and subsequently reused in another reaction. For example, the catalyst may be readily separated from a reaction mixture after use and may, in some embodiments, be efficiently recycled more than once, more than 5 times, more than 10 times, or greater, without substantially no change in catalytic performance relative to the first use.

The present invention relates to the discovery that optically active alpha-aminonitriles may be produced in high yield and with high optical purity using an efficient catalyst and related methods involving lower amounts of catalyst and shorter reaction times relative to previous methods. Optically active alpha-aminonitriles are useful intermediates in the synthesis of pharmaceuticals, fine chemicals, and the like. In some embodiments, optically active alpha-aminonitriles are useful intermediates in the synthesis of alpha-amino acids. In a particular set of embodiments, the invention relates to the asymmetric cyanation of imines for the synthesis of optically active alpha-aminonitriles using a titanium catalyst comprising an optically active ligand such as tridentate N-salicyl-beta-aminoalcohol, for example. As described herein, the present invention provides titanium catalysts for asymmetric synthesis reactions. The titanium catalyst may be produced by combining a titanium alkoxide and a ligand (e.g., an optically active ligand) to form a titanium alkoxide-ligand precatalyst, which may then be brought into contact with water to form the titanium catalyst.

The following terms refer to any groups mentioned in the present invention unless otherwise indicated.

The term “alkyl group” refers to a linear, branched or cyclic alkyl group having 1 to 20 carbon atoms. In one embodiment of the present invention, the alkyl group may have 1 to 15 carbon atoms, for example 1 to 10 carbon atoms. Examples of linear alkyl groups may include, but are not limited to, a methyl group, an ethyl group, a n-propyl group, a n-butyl group, a n-pentyl group, a n-hexyl group, a n-heptyl group, a n-octyl group, a nonyl group, a n-decyl group and the like. Examples of branched alkyl groups may include, but are not limited to, an isopropyl group, an isobutyl group, a sec-butyl group, a tert-butyl group, a 2-pentyl group, a 3-pentyl group, an isopentyl group, a neopentyl group, an amyl group and the like. Examples of cyclic alkyl groups may be, but are not limited to, a cyclopropyl group, a cyclobutyl group, cyclopentyl group, a cyclohexyl group, a cycloheptyl group, a cycloctyl group and the like.

The term “alkenyl group” refers to a linear, branched or cyclic alkenyl group having 2 to 20 carbon atoms, for example 1 to 10 carbon atoms, wherein at least one carbon-carbon double bond is present. Examples of an alkenyl group may include, but are not limited to, a vinyl group, an allyl group, a crotyl group, a cyclohexenyl group, an isopropenyl group and the like.

The term “alkynyl group” refers to an alkynyl group having 2 to 20 carbon atoms, for example 2 to 10 carbon atoms, wherein at least one carbon-carbon triple bond is present. Examples may include, but are not limited to, an ethynyl group, a 1-propynyl group, a 2-propynyl group, a 1-butynyl group, a 1-pentynyl group and the like.

The term “alkoxy” refers to a linear, branched or cyclic alkoxy group having 1 to 20 carbon atoms, for example 1 to 10 carbon atoms, wherein an alkyl group is bonded to a negatively charged oxygen atom. Examples may include, but are not limited to, a methoxy group, an ethoxy group, a n-propoxy group, an isopropoxy group, a n-butoxy group, a cyclopentyloxy group, a cyclohexyloxy group, a menthyloxy group and the like.

The term “aryl group” refers to an aryl group referring to any functional group or substituent derived from a simple aromatic ring having 6 to 20 carbon atoms. In one embodiment of the present invention, the aryl group may have 6 to 10 carbon atoms. Examples may include, but are not limited to, a phenyl group, a napththyl group, a biphenyl group, an anthryl group and the like.

The term “aryloxy group” refers to an aryloxy group having 6 to 20 carbon atoms, for example 6 to 10 carbon atoms, wherein an aryl group is bonded to a negatively charged oxygen atom. Examples may include, but are not limited to, a phenoxy group, a naphthyloxy group and the like.

The term “aromatic heterocyclic group” refers to an aromatic heterocyclic group having 3 to 20 carbon atoms, for example 1 to 10 carbon atoms, wherein at least one carbon atom of the aromatic group is replaced by a heteroatom such as nitrogen, oxygen or sulfur. Examples may include, but are not limited to, an imidazolyl group, a furyl group, a thienyl group, a pyridyl group and the like.

The term “non-aromatic heterocyclic group” refers to a non-aromatic heterocyclic group having 4 to 20 carbon atoms, for example 4 to 10 carbon atoms, wherein at least one carbon atom of the non-aromatic group is replaced by a hetero atom such as nitrogen, oxygen or sulfur. Examples may include, but are not limited to, a pyrrolidyl group, a piperidyl group, a tetrahydrofuryl group and the like.

The term “acyl group” refers to an alkylcarbonyl group having 2 to 20 carbon atoms, for example 1 to 10 carbon atoms and an arylcarbonyl group having 6 to 20 carbon atoms, for example 1 to 10 carbon atoms.

The term “alkylcarbonyl group” refers to, but is not limited to, an acetyl group, a propionyl group, a butyryl group, an isobutyryl group, a pivaloyl group and the like.

The term “arylcarbonyl group” refers to, but is not limited to, a benzoyl group, a napththoyl group, an anthrylcarbonyl group and the like.

The term “alkoxycarbonyl group” refers to a linear, branched or cyclic alkoxycarbonyl group having 2 to 20 carbon atoms, for example 2 to 10 carbon atoms. Examples may include, but are not limited to, a methoxycarbonyl group, an ethoxycarbonyl group, a n-butoxycarbonyl group, a n-octyloxycarbonyl group, an isopropoxycarbonyl group, a tert-butoxycarbonyl group, a cyclopentyloxycarbonyl group, a cyclohexyloxycarbonyl group, a cyclooctyloxycarbonyl group, an L-menthyloxycarbonyl group, a D-menthyloxycarbonyl group and the like.

The term “aryloxycarbonyl group” refers to an aryloxycarbonyl group having 7 to 20 carbon atoms, for example 7 to 15 carbon atoms. Examples may include, but are not limited to, a phenoxycarbonyl group, alpha-naphthyloxycarbonyl group and the like.

The term “aminocarbonyl group” refers to an aminocarbonyl group having a hydrogen atom, an alkyl group, an aryl group, and two of the substituents other than a carbonyl group to be bonded to a nitrogen atom may be linked together to form a ring. Examples may include, but are not limited to an isopropylaminocarbonyl group, a cyclohexylaminocarbonyl group, a tert-butylaminocarbonyl group, a tert-amylaminocarbonyl group, a dimethylamino carbonyl group, a diethylaminocarbonyl group, diisopropylaminocarbonyl group, a diisobutylaminocarbonyl group, a dicyclohexylaminocarbonyl group, a tert-butylisopropylamino carbonyl group, a phenylaminocarbonyl group, a pyrrolidylcarbonyl group, a piperidylcarbonyl group, an indolecarbonyl group and the like.

The term “amino group” refers to organic compounds and a type of functional group that contain nitrogen as the key atom. The term refers to an amino group having a hydrogen atom, a linear, branched or cyclic alkyl group, or an amino group having an aryl group. Two substituents to be bonded to a nitrogen atom may be linked together to form a ring. Examples of the amino group having an alkyl group or an aryl group may include, but are not limited to, an isopropylamino group, a cyclohexylamino group, a tert-butylamino group, a tert-amylamino group, a dimethylamino group, a diethylamino group, a diisopropylamino group, a diisobutylamino group, a dicyclohexylamino group, a tert-butylisopropylamino group, a pyrrolidyl group, a piperidyl group, an indole group and the like.

The term “halogen atom” refers to F, Cl, Br, I, and the like.

The term “silyl group” refers to a silyl group having 2 to 20 carbon atoms,

wherein the silyl group can be considered as silicon analogue of an alkyl. Examples may include, but are not limited to, a trimethylsilyl group, a tert-butyldimethylsilyl group and the like.

The term “siloxy group” refers to a siloxy group having 2 to 20 carbon atoms. Examples may include, but are not limited to, a trimethylsiloxy group, a tert-butyldimethylsiloxy group, a tert-butyldiphenylsiloxy group and the like.

All of the above mentioned groups may optionally have one or more substituents. “Having one or more substituents” in the context of the present invention means that at least one hydrogen atom of the above compounds may be replaced by F, Cl, Br, I, OH, CN, NO₂, NH₂, SO₂, an alkyl group, an aryl group, an aromatic heterocyclic group, a non-aromatic heterocyclic group, an oxygen containing group, a nitrogen containing group, a silicon containing group or the like.

Examples of the oxygen containing group may include, but are not limited to, those having 1 to 20 carbon atoms such as an alkoxy group, an aryloxy group, an alkoxycarbonyl group, an aryloxycarbonyl group, an acyloxy group and the like. Examples of the nitrogen containing group may include, but are not limited to, an amino group having 1 to 20 carbon atoms, an amide group having 1 to 20 carbon atoms, a nitro group, a cyano group and the like. Examples of the silicon containing group may include, but are not limited to, those having 1 to 20 carbon atoms such as a silyl group, a silyloxy group and the like.

Examples of substituted alkyl groups may include, but are not limited to, a chloromethyl group, a 2-chloro ethyl group, a trifluoromethyl group, a 2,2,2-trifluoroethyl group, a perfluoroethyl group, a perfluorohexyl, a substituted or unsubstituted aralkyl group such as a benzyl group, a diphenylmethyl group, a trityl group, a 4-methoxybenzyl group, a 2-phenylethyl group, a cumyl group, an alpha-napthylmethyl, a 2-pyridylmethyl group, a 2-furfuryl group, a 3-furfuryl group, a 2-thienylmethyl group, a 2-tetrahydrofurfuryl group, a 3-tetrahydrofurfuryl group, a methoxymethyl group, a methoxyethyl group, a phenoxyethyl group, an isopropoxymethyl group, a tert-butoxymethyl group, a cyclohexyloxymethyl group, a L-menthyloxymethyl group, a D-menthyloxymethyl group, a phenoxymethyl group, a benzyloxymethyl group, a phenoxymethyl group, an acetyloxymethyl group, a 2,4,6-trimethylbenzoyloxymethyl, a 2-(dimethylamino)ethyl group, a 3-(diphenylamino)propyl group, a 2-(trimethylsiloxy)ethyl group and the like.

Examples of substituted alkenyl groups may include, but are not limited to, a 2-chlorovinyl group, a 2,2-dichlorovinyl group, a 3-chloroisopropenyl group and the like.

Examples of substituted alkynyl groups may include, but are not limited to, a 3-chloro-1-propynyl group, a 2-phenylethynyl group, a 3-phenyl-2-propynyl group, a 2-(2-pyridylethynyl) group, a 2-tetrahydrofurylethynyl group, a 2-methoxyethynyl group, a 2-phenoxyethynyl group, a 2-(dimethylamino)ethynyl group, a 3-(diphenylamino)propynyl group, a 2-(trimethylsiloxy)ethynyl group and the like.

Examples of substituted alkoxy groups may include, but are not limited to, a 2,2,2-trifluoroethoxy group, a benzyloxy group, a 4-methoxybenzyloxy group, a 2-phenylethoxy group, a 2-pyridylmethoxy group, a furfuryloxy group, a 2-thienylmethoxy group, a tetahydrofurfuryloxy group and the like.

Examples of substituted aryl groups may include, but are not limited to, a 4-fluorophenyl group, a pentafluorophenyl group, a tolyl group, a dimethylphenyl group such as a 3,5-dimethylphenyl group, a 2,4,6-trimethylphenyl group, a 4-isopropylphenyl group, a 3,5-diisopropylphenyl group, a 2,6-diisopropylphenyl group, a 4-tert-butylphenyl group, a 2,6-di-tert-butylphenyl group, a 4-methoxyphenyl group, a 3,5-dimethoxyphenyl group, a 3,5-diisopropoxyphenyl group, a 2,4,6-triisopropoxyphenyl group, a 2,6-diphenoxyphenyl group, a 4-(dimethylamino)phenyl group, a 4-nitrophenyl group, 3,5-bis(trimethylsilyl)phenyl group, a 3,5-bis(trimethylsiloxy)phenyl group and the like.

Examples of substituted aryloxy groups may include, but are not limited to, a pentafluorophenoxy group, a 2,6-dimethylphenoxy group, a 2,4,6-trimethylphenoxy group, a 2,6-dimethoxyphenoxy group, a 2,6-diisopropoxyphenoxy group, a 4-(dimethylamino)phenoxy group, a 4-cyanophenoxy group, a 2,6 bis(trimethylsilyl)phenoxy group, a 2,6-bis(trimethylsiloxy)phenoxy group and the like.

Examples of substituted aromatic heterocyclic groups may include, but are not limited to, an N-methylimidazolyl group, a 4,5-dimethyl-2-furyl group, a 5-butoxycarbonyl-2-furyl group, a 5-butylaminocarbonyl-2-furyl group, and the like.

Examples of substituted non-aromatic heterocyclic groups may include, but are not limited to, a 3-methyl-2-tetrahydrofuranyl group, a N-phenyl-4-piperidyl group, a 3-methoxy-2-pyrrolidyl group and the like.

Examples of substituted alkylcarbonyl group may include, but are not limited to, a trifluoroacetyl group and the like.

Examples of substituted arylcarbonyl groups may include, but are not limited to, a pentafluorobenzoyl group, a 3,5-dimethylbenzoyl group, a 2,4,6-trimethylbenzoyl group, a 2,6-dimethoxybenzoyl group, a 2,6-diisopropoxybenzoyl group, a 4-(dimethylamino)benzoyl group, a 4-cyanobenzoyl group, a 2,6-bis(trimethylsilyl)benzoyl group, a 2,6-bis(trimethylsiloxy)benzoyl group and the like.

Examples of the alkoxycarbonyl group having a halogen atom include a 2,2,2-trifluoroethoxycarbonyl group, a benzyloxycarbonyl group, a 4-methoxybenzyloxycarbonyl group, a 2-phenylethoxycarbonyl group, a cumyloxycarbonyl group, an alpha-naphthylmethoxycarbonyl group, a 2-pyridylmethoxycarbonyl group, a furfuryloxycarbonyl group, a 2-thienylmethoxycarbonyl group, a tetrahydrofurfuryloxycarbonyl group, and the like.

Examples of substituted aryloxycarbonyl groups may include, but are not limited to, a pentafluorophenoxycarbonyl group, a 2,6-dimethylphenoxycarbonyl group, a 2,4,6-trimethylphenoxycarbonyl group, a 2,6-dimethoxyphenoxycarbonyl group, a 2,6-diisopropoxyphenoxycarbonyl group, a 4-(dimethylamino)phenoxycarbonyl group, a 4-cyanophenoxycarbonyl group, a 2,6-bis(trimethylsilyl)phenoxycarbonyl group, a 2,6-bis(trimethylsiloxy)phenoxycarbonyl group and the like.

Examples of substituted aminocarbonyl groups may include, but are not limited to, a 2-chloroethylaminocarbonyl group, a perfluoroethylaminocarbonyl group, a 4-chlorophenylaminocarbonyl group, a pentafluorophenylaminocarbonyl group, a benzylaminocarbonyl group, a 2-phenylethylaminocarbonyl group, an alpha-naphthylmethylamino carbonyl and a 2,4,6-trimethylphenylamino carbonyl group and the like.

Examples of substituted amino groups may include, but are not limited to, a 2,2,2-trichloroethylamino group, a perfluoroethylamino group, a pentafluorophenylamino group, a benzylamino group, a 2-phenylethylamino group, an alpha-naphthylmethylamino and a 2,4,6-trimethylphenylamino group and the like.

In one aspect, the present invention relates to titanium compounds (e.g., catalysts) for synthesis reactions, such as cyanation of imines. The titanium catalyst may be produced by contacting a reaction mixture comprising a titanium alkoxide and a ligand (e.g., an optically active ligand) with water. The reaction mixture comprising the titanium alkoxide and the ligand may be obtained by combining a titanium alkoxide, a ligand, and optional additional components, such as solvents, additives, and the like. In some embodiments, the titanium alkoxide may associate with the ligand and form a titanium alkoxide-ligand precatalyst, i.e., a “precatalyst.” As used herein, a “precatalyst” may refer to a chemical species which, upon activation, may produce an active catalyst species in a reaction. For example, the titanium alkoxide-ligand precatalyst may be contacted with water to form a titanium catalyst (e.g., a titanium cluster compound). As used herein, the term “catalyst” includes active forms of the catalyst participating in the reaction as well as catalyst precursors (e.g., precatalysts) that may be converted in situ into the active form of the catalyst.

In some embodiments, the titanium alkoxide used in the preparation of the titanium catalyst may be a compound represented by the general formula (d),

Ti(OR′)ₓY₍₄₋ₓ₎ (d)

wherein R′ is an alkyl group, an alkenyl group, or an aryl group, each of which may have a substituent; Y can be the same or different and is a halogen atom, an acyl group, or an acetylacetonate group; and x is an integer having a value of 0-4. In one embodiment, x is 4. In some embodiments, R′ is an alkyl group, such as ethyl, n-butyl, n-propyl, iso-propyl, and the like. For example, the titanium alkoxide used may be Ti(OMe)₄, Ti(OEt)₄, Ti(On-Pr)₄, Ti(Oi-Pr)₄, Ti(On-Bu)₄, TiCl(Oi-Pr)₃, or [EtOCOCH═C(O)Me]₂Ti(Oi-Pr)₂. In some embodiments, R′ is an aryl group.

The titanium compound (e.g., catalyst) of the present invention may be produced by contacting water with a titanium alkoxide-ligand precatalyst obtained by combining a titanium alkoxide monomer with a ligand. The ligand may be represented by the general formula (e),

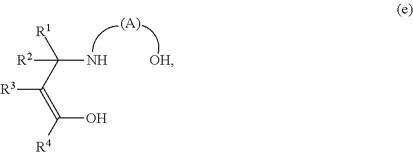

wherein R¹, R², R³, and R⁴ are independently a hydrogen atom, an alkyl group, an alkenyl group, an aryl group, an aromatic heterocyclic group, a non-aromatic heterocyclic group, an acyl group, a hydroxyl group, an alkoxy group, an aryloxy group, an amino group, a cyano group, a nitro group, a silyl group, a siloxy group, an alkoxycarbonyl group or an aryloxycarbonyl group, each of which may have a substituent, or two or more of R¹, R², R³, and R⁴ may be linked together to form a ring, and the ring may have a substituent; and (A) represents a group with two or more carbon atoms. In some embodiments, R¹, R², R³, and R⁴ are independently a hydrogen atom, a halogen atom, an alkyl group, an alkenyl group, an aryl group, an aromatic heterocyclic group, a non-aromatic heterocyclic group, an acyl group, an alkoxycarbonyl group, or an aryloxycarbonyl group, each of which may have a substituent, or two or more of R¹, R², R³, and R⁴ may be linked together to form a ring, and the ring may have a substituent.

In some embodiments, the ligand may be an optically active ligand. In some cases, the optically active ligand may be represented by the general formula (a),

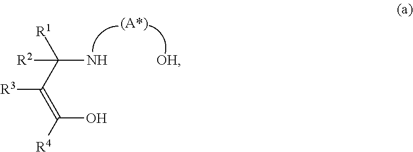

wherein R¹, R², R³, and R⁴ are as described herein; and (A*) represents a group with two or more carbon atoms having an asymmetric carbon atom or axial asymmetry.

In some cases, R¹, R², R³, or R⁴ may be an alkyl group, optionally having one or more substituents. Furthermore, two or more of R¹, R², R³, and R⁴ may be linked together to form a ring. The ring may be an aliphatic or aromatic hydrocarbon ring. The formed rings may be condensed to form a ring, respectively. In some embodiments, the aliphatic hydrocarbon ring is a 10- or less-membered ring, such as a 3- to 7-membered ring, or a 5- or 6-membered ring. The aliphatic hydrocarbon ring may have unsaturated bonds. The aromatic hydrocarbon ring may be a 6-membered ring, such as a phenyl ring. For example, when two of R¹, R², R³, and R⁴ are linked together to form —(CH₂)₄— or —CH═CH—CH═CH—, a cyclohexene ring (included in the aliphatic hydrocarbon ring) or a phenyl ring (included in the aromatic hydrocarbon ring) may be formed, respectively. The ring may have one or more substituents, including a halogen atom, an alkyl group, an aryl group, an alkoxy group, an aryloxy group, an amino group, a nitro group, a cyano group, a silyl group and a silyloxy group, and the like.

In one set of embodiments, R¹ and R² are hydrogen atoms, and R³ and R⁴ are linked together to form a phenyl ring, wherein the phenyl ring may have one or more substituents.

In the general formula (a), (A*) represents an optically active group with two or more carbon atoms, and preferably 2 to 40 carbon atoms, having an asymmetric carbon atom or axial asymmetry which may have a substituent. Examples of (A*) include the following structures,

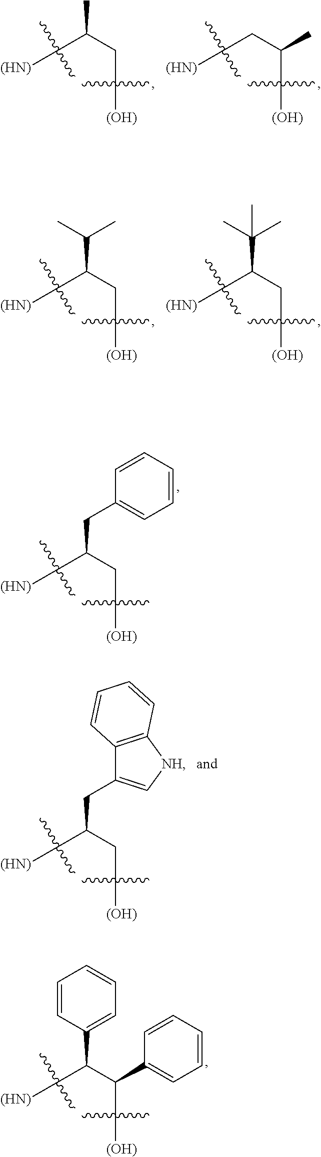

wherein parts indicated as (N) and (OH) do not belong to (A*), and represent an amino group and a hydroxyl group, respectively, corresponding to those in the above general formula (a) to which (A*) is bonded.

In some cases, the optically active ligand is represented by the general formula (b),

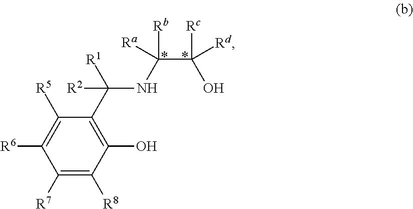

wherein R^{a}, R^{b}, R^{c}, and R^{d} are each a hydrogen atom, an alkyl group, an aryl group, alkoxycarbonyl group, an aryloxycarbonyl group or an aminocarbonyl group, each of which may have a substituent, or two or more of R^{a}, R^{b}, R^{c}, and R^{d} may be linked together to form a ring, and the ring may have a substituent; at least one of R^{a}, R^{b}, R^{c}, and R^{d} is a different group; both or at least one of the carbon atoms indicated as * become an asymmetric center; and parts indicated as (NH) and (OH) do not belong to (A*), and represent an amino group and a hydroxyl group, respectively, corresponding to those in said general formula (a) to which (A*) is bonded; R⁵, R⁶, R⁷, and R⁸ are independently a hydrogen atom, a halogen atom, an alkyl group, an alkenyl group, an aryl group, an aromatic heterocyclic group, a non-aromatic heterocyclic group, an alkoxycarbonyl group, an aryloxycarbonyl group, a hydroxyl group, an alkoxy group, an aryloxy group, an amino group, a cyano group, a nitro group, a silyl group or a siloxy group which may have a substituent, each of which may be linked together to form a ring.

In some cases, R^{a} is methyl, ethyl, n-propyl, iso-propyl, n-butyl, iso-butyl, sec-butyl, tert-butyl, or benzyl, and R^{b}, R^{c}, and R^{d} are hydrogen atoms.

Examples of the optically active ligand include, but are not limited to,

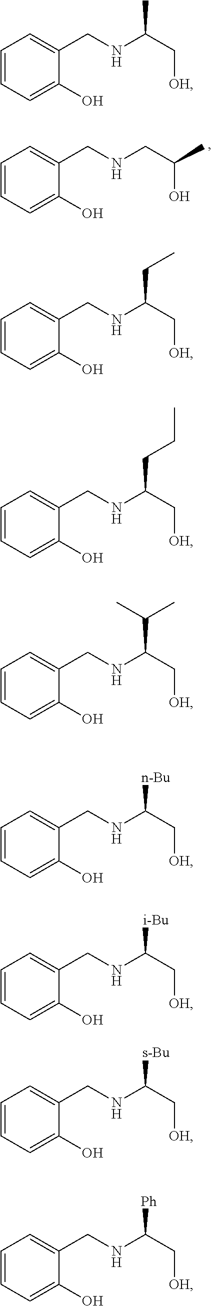

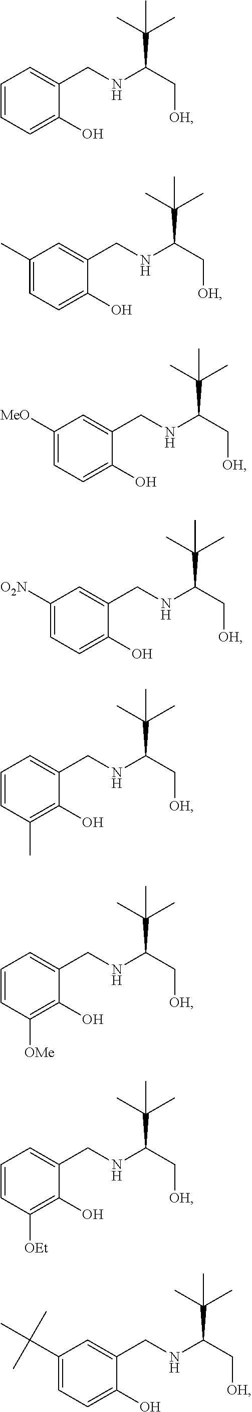

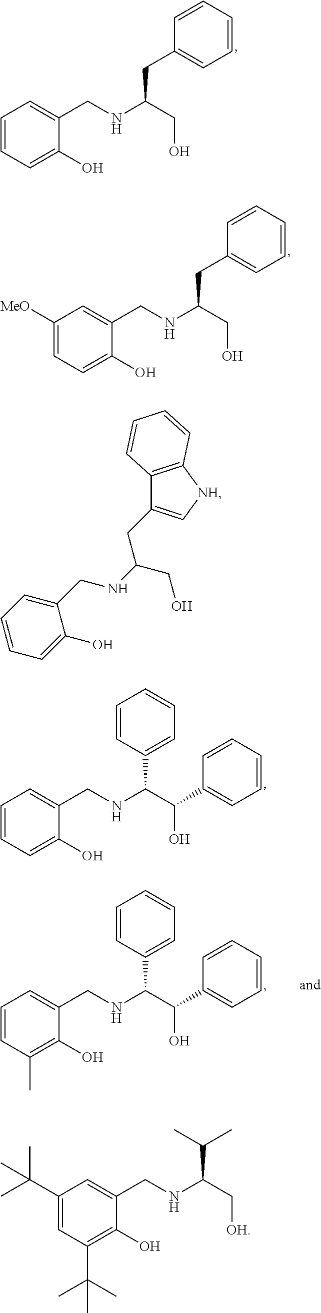

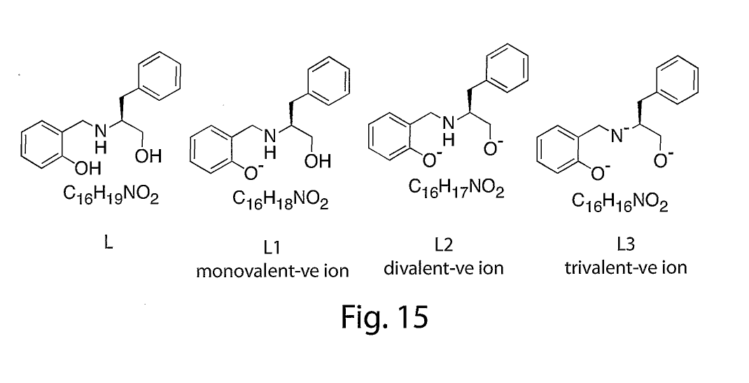

In some cases, upon coordination of ligand L to titanium alkoxide, the ligand may become deprotonated at one or more locations of the ligand, thereby forming an ionic species. In some embodiments, the ligand L may be a monovalent ion, a divalent ion, or a trivalent ion. In one set of embodiments, the ligand L is a divalent ion. For example, a proton from one hydroxyl groups may be lost and the ligand L may exist in the complex as a monovalent ion. In some cases, protons from two hydroxyl groups may be lost and the ligand L may exist in the complex as a divalent ion. In another set of embodiments, protons from two hydroxyl groups and from an amine group may be lost and the ligand L may exist in the complex as a trivalent ion. FIG. 15 shows an illustrative embodiment of a ligand L that may exist in a titanium complex as a monovalent ion (L1), a divalent ion (L2), or a trivalent ion (L3).

A titanium catalyst of the present invention can be produced by bringing a reaction mixture comprising a titanium alkoxide and a ligand represented by the general formula (e) or (a), as described above into contact with, for example, water. The preparation of the titanium catalyst may further comprise use of a solvent, such as an organic solvent. For example, the reaction mixture may be obtained by combining a titanium alkoxide and a ligand in an organic solvent. The molar ratio of the titanium alkoxide, water, and the ligand represented by general formula (e) or (a) can be in the range of 1.0:0.1:0.1 to 1.0:3.0:3.0. Any molar ratio within this range may be suitable for use in the present invention.

In some embodiments, the process for the preparation of a titanium catalyst comprises the steps of i) forming a solution comprising of a complex prepared from a titanium alkoxide represented by the general formula (d) (e.g., as described herein) and a ligand represented by the general formula (e) or (a) (e.g., as described herein), ii) contacting water with the solution to give solution or suspension of the catalyst, and removing the solvent from the solution or suspension of the catalyst. The above steps are discussed more herein.

It should be noted that titanium catalysts comprising a ligand represented by the general formula (a) are discussed herein by way of example only, and other ligands (e.g., ligands represented by the general formula (e) or (b)) may be used in the context of the invention.

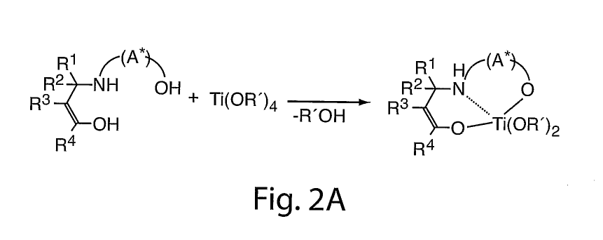

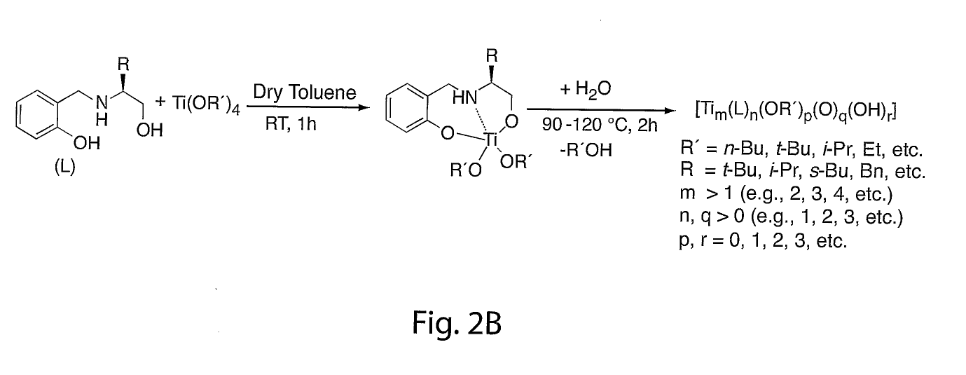

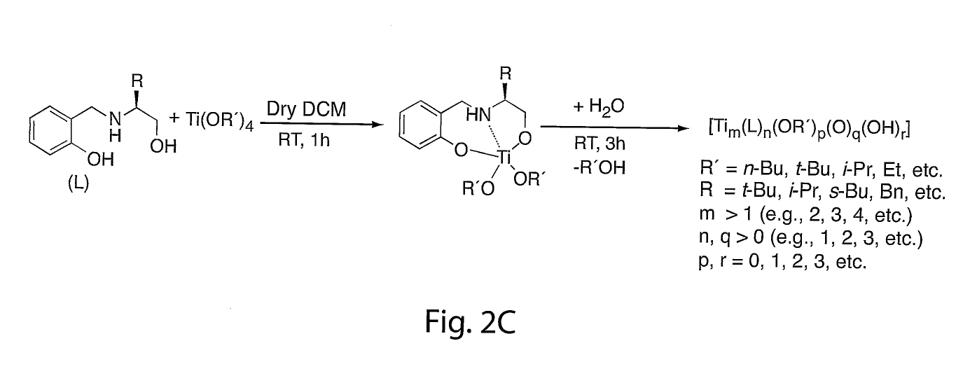

In some embodiments, the titanium catalyst is prepared by first reacting a titanium alkoxide (e.g., a titanium tetraalkoxide) compound with a ligand represented by general formula (a) or (e) in an organic solvent to form a titanium alkoxide-ligand precatalyst. A titanium-alkoxide precatalyst may comprise a titanium atom, at least one ligand represented by the general formula (a) or (e), and one or more alkoxides (e.g., titanium alkoxides used to prepare the precatalyst). In some embodiments, the titanium alkoxide-ligand precatalyst may be a monomeric titanium alkoxide-ligand precatalyst, wherein the precatalyst comprises one titanium atom, one ligand associated with the titanium atom and at least two alkoxides associated with the titanium atom. FIG. 2A shows a non-limiting example of the formation of a monomeric titanium alkoxide-ligand precatalyst. The molar ratio of titanium alkoxide to ligand can be in the range of 1:0.1 to 1:3. In some embodiments, the molar ratio is about 1.0 to about 1.0, about 1.0 to about 2.0, or about 2.0 to about 1.0.

Examples of organic solvents suitable for use in the invention include halogenated hydrocarbon solvents such as dichloromethane, chloroform, fluorobenzene, trifluoromethylbenzene and the like; aromatic hydrocarbon solvents such as toluene, xylene and the like; ester solvents such as ethyl acetate and the like; and ether solvents such as tetrahydrofuran, dioxane, diethyl ether, dimethoxyethane and the like. In some embodiments, halogenated solvents or aromatic hydrocarbon solvents are used. The total amount of the solvent used in combining the titanium alkoxide and the ligand are mixed may be from about 1 to 500 mL, or from about 10 to 50 mL, based on 1 millimole of the titanium alkoxide.

The temperature at which the titanium alkoxide is combined with the ligand may be any temperature which does not freeze the solvent. For example, the reaction may be carried out at about room temperature, for example, from 15 to 30° C. The reaction may also be carried out at higher temperatures (e.g., by heating) depending on the boiling point of the solvent in use. The time required for the reaction is different depending on general conditions such as the amount of water to be added, the reaction temperature, and the like. In some embodiments, the time required for stirring is about 1 hour to achieve formation of the titanium alkoxide-ligand precatalyst.

Next, the titanium alkoxide-ligand precatalyst may then be contacted with a quantity of a hydrolyzing agent, such as water, to form a titanium catalyst. FIGS. 2B and 2C show non-limiting examples of the synthesis of titanium catalysts via a titanium alkoxide-ligand precatalyst. At the time of addition, the titanium alkoxide-ligand precatalyst may be dissolved and/or suspended in a solvent and/or stirred while water is added. In some cases, the titanium alkoxide-ligand precatalyst is dissolved in an organic solvent and stirred while water is added. Water may be added directly to the mixture (e.g., solution, suspension, etc.) or may be diluted in a solvent (e.g., THF), prior to addition. When a solvent is used, the solvent can be the same or different from the solvent used in the above step combining the titanium alkoxide and the ligand. Water may be directly added using various methods, including addition of water in mist form, and/or use of a reaction vessel equipped with a high efficiency stirrer, or the like. It should be understood that other aqueous solvents (e.g., alcohols, etc.) may be used in methods described herein, and the use of water is described by way of example only.

Any of the solvents described herein may be added in an amount from about 1 to about 5.0 mL, or from about 1 to about 500 mL, based on 1 millimole of the titanium atom. Water may be added to the titanium alkoxide-ligand precatalyst over a period of time, for example, between about 1 second and about 1 hour, or any suitable time within that range. In some cases, water is added over a period of about 15 minutes. The quantity of water added may be based on the amount of titanium alkoxide provided in the first step of the reaction. For example, the quantity of water added may be such that the ratio of titanium alkoxide to water is from 1:0.1 to 1:3, or any molar ratio within that range.

In some cases, water may be added to the titanium alkoxide-ligand precatalyst by providing a hydrated material (e.g., to a solution comprising the precatalyst). A “hydrated material,” as used herein, is a material which comprises water, such as absorbed water. The hydrated material may be any material that is capable of comprising absorbed water and/or capable of releasing the absorbed water (e.g., upon exposure to a solution comprising a titanium alkoxide-ligand precatalyst). For example, a hydrated material may be molecular sieves comprising absorbed water. The hydrated material, in some embodiments, does not substantially interfere with the reaction components, the reaction to form the titanium catalyst, and/or the catalysis reaction.

The temperature at which the titanium alkoxide-ligand precatalyst is reacted with the water may be any temperature which does not freeze the solvent. For example, the reaction may be carried out at about room temperature, for example, from 15 to 30° C. The reaction may also be carried out at higher temperatures (e.g., by heating) depending on the boiling point of the solvent in use. Those of ordinary skill in the art would be able to select the appropriate temperature in combination with the solvent being utilized. In some cases, the titanium catalyst may be produced by stirring at above room temperature, for example, between about 15° C. and about 150° C. After addition of water, the reaction may be stirred for about 5 minutes to about 5 hour, or from about 1 minute to about 3 hour at the desired temperature. In some cases, the reaction may be stirred for about 1 hour, about 2 hours, about 3 hours, or about 4 hours.

After addition of water and formation of the titanium catalyst, the titanium catalyst may be isolated. That is, the catalyst may be isolated substantially free of any solvent and/or impurities. In some cases, following addition of water, the volatile components (e.g., water, solvent, alcohol released during formation of the catalyst, etc.) may be removed from the catalyst to produce a solid catalyst. In some cases, the catalyst may be isolated by removing the solvent from the solution or suspension of the catalyst. The volatile components (e.g., solution) may be removed using methods known to those of ordinary skill in the art, for example, by evaporation (e.g., with or without heating), azeotroping, drying under vacuum, and/or by filtration of a suspension. The titanium catalyst may be isolated as a solid, a semi-solid, or a gel. In some cases, the titanium catalyst may be isolated as a solid (e.g., a powder). The titanium catalyst may or may not be washed at least once with a solvent prior to drying. The isolated titanium catalyst may be at least about 70%, about 80%, about 85%, about 90%, about 95%, about 97%, about 99%, about 99.9%, or about 100% free from impurities.

In some embodiments, the titanium catalyst comprising at least one titanium atom and at least one ligand associated with the titanium atom and represented by the general formula (a) or (e). The ligand may be associated with the titanium atom by formation of one or more bonds (e.g., covalent bond, ionic bond, hydrogen bond, etc.). In some cases, a titanium catalyst, prepared using a titanium alkoxide (e.g., Ti(OR′)ₓY₍₄₋ₓ₎), has the general formula,

[Tiₘ(L)ₙ(OR′)ₚ(O)_{q}(OH)ᵣYₛ] (f)

wherein R′ can be the same or different is a group as described herein (e.g., as defined in Ti(OR′)ₓY₍₄₋ₓ₎ above); L is a ligand represented by the general formula (a) or (e); Y can be the same or different and is a halogen atom, an acyl group, or an acetylacetonate group; m is an integer greater than 1 (e.g., 2, 3, 4, 5, 6, etc.); n and q are the same or different and are 0 or an integer greater than 0 (e.g., 1, 2, 3, 4, 5, 6, etc.); p, r and s are the same or different and either zero or an integer greater than 0 (e.g., 0, 1, 2, 3, 4, 5, 6, etc.). In some cases, one or more ligands (e.g., (O), (OH), (OR′), etc.) may form a bridge between two or more titanium atoms.

In some embodiments, the titanium catalyst comprises a compound as represented by any one of the following formulas:

[Tiₘ(L)ₙ(OH)ᵣ] (g),

[Tiₘ(L)ₙ(O)_{q}(OH)ᵣ] (h), or

[Tiₘ(L)ₙ(OR′)ₚ(OH)ᵣ] (i).

The titanium catalyst may comprise any number of titanium atoms, for example, 1, 2, 3, 4, etc. The isolated titanium catalyst may be represented by the one or more of the following non-limiting formulas: Ti₂L₂O₂(OR′)₂, Ti₂L₂O₂(OH)(OR′)₂, TiL₂(OR′)₄, Ti₃L₃O₃(OH)₂, Ti₃L₃(OH)₆, Ti₃L₃(O)₃(OH)₃, Ti₃L₂(OR′)₃(OH)₃, and Ti₃L₃O₃(OR′)₂, wherein R′ represents the R′ from the Ti(OR′)ₓY₍₄₋ₓ₎ compound used to form the catalyst and L represents the general structure of a ligand represented by the general formula (a), for example,

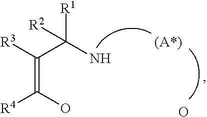

wherein (A*), R¹, R², R³, and R⁴ are described herein.

The isolated titanium catalyst may comprise a mixture of titanium catalysts having different chemical formulas. For example, the isolated titanium catalyst may comprise at least 2, at least 3, at least 4, at least 5, or at least 6 different types of titanium catalysts, i.e., catalysts having different chemical formulas. The titanium atoms in a titanium catalyst may associate with additional atoms in the ligand. For example, a titanium atom may associate with the nitrogen atom from the ligand represented by the general formula (a) or (e).

In some cases, the production of a titanium compound of the present invention may advantageously be carried out under ambient conditions. However, it should also be understood that the production of the titanium compound of the present invention may be carried out under a dry and/or inert gas atmosphere or without strictly following dry and inert conditions. Examples of the inert gas include nitrogen, argon, helium and the like.

In some embodiments, the titanium catalyst may be recycled. For example, a titanium catalyst may be used in a first reaction for the cyanation of imines, and then may be isolated and used in at least a second reaction for the cyanation of imines, or other reaction. The catalyst may be reused at least 2, at least 3, at least 4, at least 5, at least 6, at least 7, at least 8, at least 9, at least 10, at least 15, at least 20, at least 30, at least 40, at least 50, or more, times for in a reaction for the cyanation of imines, with minimal or essentially no difference or decrease in the catalyst performance. That is, the results (e.g., yield, enantiomeric excess, etc.) of the cyanation of an imine in a first reaction may not be substantially different than that of a second reaction using the catalyst, isolated from the first reaction (e.g., the catalyst performance may not significantly degrade between a first use and at least a second use). In some cases, the yield and/or the enantiomeric excess of the cyanation of an imine may be about 100%, about 99.9%, about 99.7%, about 99.5%, about 99%, about 98%, about 97%, about 96, about 95%, about 90%, of the yield and/or enantiomeric excess of a prior cyanation reaction using the same catalyst, under essentially identical conditions.

The titanium catalyst may be isolated from a reaction mixture using methods known to those of ordinary skill in the art. For example, a catalyst may be isolated by centrifuging the reaction mixture and then removing the non-solid components (e.g., fluids) by decanting, pipetting, or any other common technique. As another example, the catalyst may be isolated by filtration. In some cases, the catalyst may be washed at least once prior to used in subsequent reactions.

As described herein, one or more solvents may be used in the preparation of the titanium catalyst. In some cases, use of a solvent may facilitate formation of the titanium compound. The solvent may be selected to dissolve any one of the titanium alkoxide, ligand, other component, or combinations thereof to facilitate formation of the catalyst. Examples of the solvent include halogenated hydrocarbon solvents such as dichloromethane, chloroform and the like; halogenated aromatic hydrocarbon solvents such as chlorobenzene, o-dichlorobenzene, fluorobenzene, trifluoromethylbenzene and the like; aromatic hydrocarbon solvents such as toluene, xylene and the like; ester solvents such as ethyl acetate and the like; and ether solvents such as tetrahydrofuran, dioxane, diethyl ether, dimethoxyethane and the like. In some embodiments, halogenated hydrocarbon solvents or aromatic hydrocarbon solvents may be used. In some embodiments, mixtures of the above solvents may also be used.

As described herein, some embodiments provide an isolated solid catalyst. In some embodiments, the solid catalyst may comprise particles, or aggregates of particles (e.g., microspheres, nanospheres). The particles may exhibit one or more morphologies, including a spherical morphology. In some embodiments, the solid catalyst may comprise particles having a substantially spherical shape. In some embodiments, the solid catalyst may comprise microspheres. As used herein, the term “microspheres” refers to particles having an average particle size of about 100 nm or greater. In some embodiments, the solid catalyst may comprise nano spheres. As used herein, the term “nanospheres” refers to particles having an average particle size of about 100 nm or less. It should be understood that the average particle size of a particle may be determined by measuring an average cross-sectional dimension (e.g., diameter for substantially spherical particles) of a representative number of particles. For example, the average cross-sectional dimension of a substantially spherical particle is its diameter; and, the average cross-sectional dimension of a non-spherical particle is the average of its three cross-sectional dimensions (e.g., length, width, thickness). The particle size may be measured using a scanning electron microscope, or other conventional techniques.

In some embodiments, the catalyst may comprise microspheres having an average particle size of about 100 nm, about 200 nm, about 300 nm, about 400 nm, about 500 nm, about 600 nm, about 700 nm, about 800 nm, about 900 nm, about 1000 nm, or greater. In some embodiments, the catalyst may comprise microspheres having an average particle size from about 100 nm to about 400 nm. In some embodiments, the catalyst may comprise microspheres having an average particle size of about 300 nm.

In some embodiments, the catalyst may comprise nanospheres having an average particle size of about 10 nm, about 20 nm, about 30 nm, about 40 nm, about 50 nm, about 60 nm, about 70 nm, about 80 nm, about 90 nm, or about 100 nm. In some embodiments, the catalyst may comprise microspheres having an average particle size from about 10 nm to about 100 nm. In some embodiments, the catalyst may comprise microspheres having an average particle size from about 10 nm to about 50 nm. In certain embodiments, the average particle size may be even smaller, i.e., less than 10 nm.

One advantageous feature of the present invention is that the titanium compounds (e.g., catalysts) produced as above can be isolated and/or stored for extended periods of time. The titanium catalyst may be stored in any form, for example, as a solid, a semi-solid, a gel, a slurry, a suspension, a solution, etc. The titanium catalyst may be stored in a container for at least about 1 day, at least about 10 days, at least about 1 month, at least about 6 months, or at least about 1 year. A titanium catalyst may be used to catalyze a reaction prior to storage and/or following storage to give substantially similar results, when the reactions (e.g., before storage and after storage) are conducted under substantially similar conditions. The titanium catalyst may be stored under ambient conditions or dry and/or inert conditions (e.g., under an inert gas such as nitrogen, argon, helium, etc.).

The titanium catalyst of the present invention may be used for the cyanation of imines. In some cases, the titanium catalysts may be used for the asymmetric cyanation of imines. In some embodiments, the enantioselectivity of the reaction may increase when using a titanium catalyst comprising an optically active ligand (e.g., a ligand represented by the general formula (a)), as opposed to an achiral ligand. In some cases, however, a titanium catalyst comprising an achiral ligand (e.g., a ligand represented by the general formula (e)) may produce enantiomerically enriched reaction products. It should be understood that asymmetric cyanation reactions are discussed herein by way of example only, and achiral cyanation reactions may also be performed using the catalysts described herein.

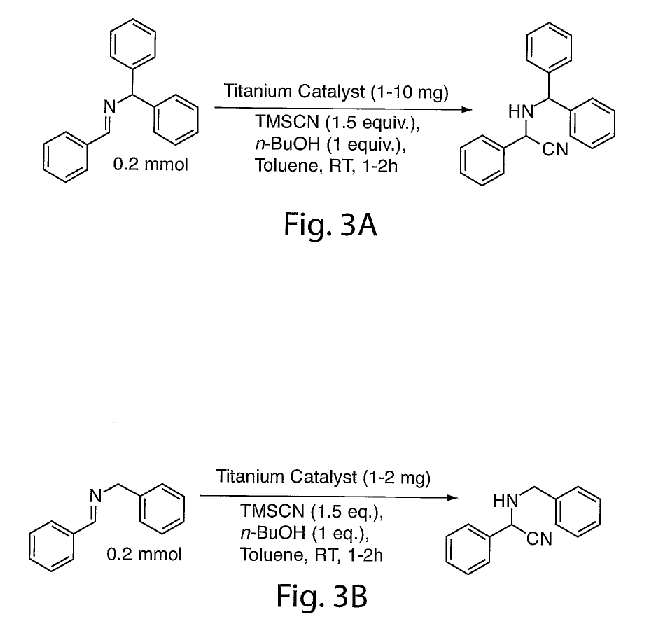

Some embodiments of the invention provide processes for producing alpha-aminonitriles (e.g., optically active alpha-aminonitriles). In methods of the invention, an imine substrate may be used as a starting material. The method may comprise reacting the imine substrate with a cyanating agent in the presence of a titanium catalyst as described herein, optionally in the presence of solvents, additives, and the like. In some cases, the imine is an unsymmetrical imine, that is, the imine has at least two different substituents on the carbon of the C═N bond. In some cases, the imine is a prochiral compound and can be suitably selected to correspond to the desired optically active alpha-aminonitrile product upon asymmetric cyanation of the imine. As a non-limiting example, FIG. 3 shows an asymmetric cyanation of a benzhydryl imine in the presence of an optically active titanium catalyst of the invention, trimethylsilyl cyanide, and n-butanol.

In some cases, processes of the invention may comprise use of an imine represented by the general formula (c),

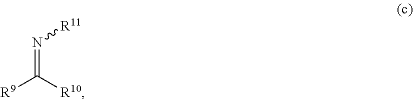

wherein R⁹ and R¹⁰ are independently a hydrogen atom, an alkyl group, an alkenyl group, an alkynyl group, an aryl group, an aromatic heterocyclic group or a non-aromatic heterocyclic group, each of which may have a substituent, and R⁹ is different from R¹⁰; R⁹ and R¹⁰ may be linked together to form a ring, and the ring may have a substituent; R¹¹ is a hydrogen atom, an alkyl group, an alkenyl group, an alkynyl group, an aryl group, an aromatic heterocyclic group or a non-aromatic heterocyclic group, a phosphonate, phosphinoyl, phosphine oxide, alkoxycarbonyl, sulfinyl or sulfoxy group, each of which may have a substituent; R¹¹ may be linked either to R⁹ or R¹⁰ to form a ring through a carbon chain, and the ring may have substituents.

In some embodiments, R⁹ is an alkyl group or an aryl group, R¹⁰ is a hydrogen atom, and R¹¹ is an alkyl group or an aryl group. In some embodiments, R⁹ is a hydrogen atom, and R¹⁰ and R¹¹ are independently an alkyl group or an aryl group.

Examples of R⁹ or R¹⁰ include, but not limited to, a hydrogen atom, phenyl, 2-chlorophenyl, 2-bromophenyl, 2-fluorophenyl, 2-methylphenyl, 2-methoxyphenyl, 4-chlorophenyl, 4-bromophenyl, 4-fluorophenyl, 4-methylphenyl, 4-methoxyphenyl, 4-trifluoromethylphenyl, 4-nitrophenyl, furanyl, pyridyl, cinnamyl, 2-phenylethyl, methyl, ethyl, n-propyl, i-propyl, n-butyl, i-butyl, s-butyl, t-butyl, pentyl, hexyl, and the like.

Examples of R¹¹ include, benzyl, benzhydryl, 9-fluorenyl, 2-hydroxyphenyl, 4-methoxyphenyl, allyl, t-butoxycarbonyl, benzyloxycarbonyl, diphenylphosphinoyl, p-tolylsulfinyl, p-toluenesulfonyl, mesitylenesulfonyl and the like. R¹¹ may also be part of a ring as in 3,4-dihydroisoquinoline, and the like.

The imine substrates described herein may be synthesized by methods known in the art, for example, by condensation of an aldehyde or ketone with an amine to produce the corresponding imine substrate.

The process involves the use of a cyanating agent as a source of cyanide ion in the cyanation reaction. Examples of cyanating agents suitable for use in the present invention include, but are not limited to, hydrogen cyanide, trialkylsilyl cyanide, acetone cyanohydrin, cyano formate ester, potassium cyanide-acetic acid, potassium cyanide-acetic anhydride, tributyltin cyanide, and the like. In some embodiments, the cyanating agent is trialkylsilyl cyanide. In some embodiments, the cyanating agent is a mixture of trialkylsilyl cyanide and hydrogen cyanide. For example, hydrogen cyanide gas may be added to the reaction vessel in combination with a solvent (e.g., as a dissolved gas in a solvent). In some cases, the cyanating agent is used in the reaction in an amount from 0.1 to 3 moles, 0.5 to 3 moles (e.g., from 0.5 to 2.5 moles), from 1 to 3 moles, from 1.05 to 2.5 moles, or, in some cases, from 1.5 to 2.5 moles, based on 1 mole of the imine substrate. In some embodiments, 1.1 equivalents of cyanating agent may be used, based on the imine substrate. In some embodiments, 1.5 equivalents of cyanating agent may be used, based on the imine substrate. The cyanating agent may be added to the reaction vessel over a period of time, such as 5 minutes to 10 hours, 10 minutes to 5 hours, or, in some cases, 30 minutes to 1 hour.

In some embodiments, the process advantageously uses inexpensive and readily available cyanating agents, such as hydrogen cyanide. For example, the process may employ hydrogen cyanide as the cyanating agent in the presence of a catalytic amount of a trialkylsilyl cyanide such as TMSCN.

As described herein, one or more solvents may be used in the cyanation of imines. Examples of the solvent include halogenated hydrocarbon solvents such as dichloromethane, chloroform and the like; halogenated aromatic hydrocarbon solvents such as chlorobenzene, o-dichlorobenzene, fluorobenzene, trifluoromethylbenzene and the like; aromatic hydrocarbon solvents such as toluene, xylene and the like; ester solvents such as ethyl acetate and the like; ester solvents such as ethyl acetate and the like; and ether solvents such as tetrahydrofuran, dioxane, diethyl ether, dimethoxyethane and the like. In some embodiments, the solvent is a halogenated hydrocarbon solvent or aromatic hydrocarbon solvent. The solvents can be used alone or in combination as a mixture of solvents. In some embodiments, the total amount of solvent used may be about 0.1-5 mL, or, in some cases, 0.2-1 mL, based on 1 mmol of imine as a substrate.

The reactions described herein may be carried out by preparing the titanium catalysts using methods as described herein, and then adding the imine substrate and cyanating agent to the titanium catalyst. The resulting mixture may be stirred at any reaction temperature, for example, from −78° C. to 80° C., or greater, for about 15 minutes to 6 hours, to produce the alpha-aminonitrile product. In some embodiments, the mixture is stirred at a reaction temperature from about 0-30° C.

In some embodiments, methods of the present invention comprise use of the titanium catalyst in reactions in an amount from 0.01 to 30 mole %, from 0.25 to 10 mole %, 2.5 to 10 mole %, or, 2.5 to 5.0 mole %, based on 1 mole of imine in terms of the titanium atom. In some embodiments, the titanium catalyst may be used in an amount that is about 1 mol % or less, based on 1 mole of imine in terms of the titanium atom. In some cases, a solid catalyst may be used in small amounts (e.g., 1 mol % or less) without a substantial change (e.g., decrease) in enantioselectivity, relative to reactions in which larger amounts of catalyst are used.

The temperature at which the cyanation reaction occurs may be any temperature which does not freeze the components of the reactions, including the catalyst, imine substrate, cyanating agent, or other optional components including solvents and additives. In some cases, the reaction may be carried out at about room temperature, for example, from 15 to 35° C. The reaction may also be carried out at higher temperatures (e.g., by heating) depending on the boiling point of the solvent in use. The time required for the reaction is different depending on general conditions such as the reaction temperature, and the like. In some cases, the reaction time is six hours or less, 4 hours or less, two hours or less, 1 hour or less, 45 minutes or less, 30 minutes or less, or in some cases, 15 minutes or less. In some embodiments, the time required for stirring is about 15-60 minutes to achieve formation of an optically active alpha-aminonitrile product in high yield and with high enantioselectivity.

In some cases, the cyanation reaction may advantageously be carried out under ambient conditions. However, it should also be understood that the production of the titanium compound of the present invention may be carried out under a dry and/or inert gas atmosphere or without strictly following dry and inert conditions. Examples of the inert gas include nitrogen, argon, helium and the like. Subsequent to stirring of the reaction mixture, the optically active alpha-aminonitrile product can be obtained.

In some embodiments, an additive may also be used in the cyanation of imines. For example, the additive may be added to the mixture comprising the titanium catalyst, the imine substrate, the cyanating agent, and/or solvent. The additive may be, for example, a species comprising at least one hydroxyl group (e.g., water, alcohols, diols, polyols, etc.). In some embodiments, the additive is water. In some embodiments, the additive is an alcohol. Examples of the alcohols suitable for use as an additive include an aliphatic alcohol and an aromatic alcohol, each of which may have a substituent, and/or combinations thereof. In some cases the alcohol is an alkyl alcohol, including linear, branched or cyclic alkyl alcohols having 10 carbon atoms or less. Some examples of alkyl alcohols include methanol, ethanol, n-propanol, isopropanol, n-butanol, sec-butanol, tert-butanol, cyclopentyl alcohol, cyclohexyl alcohol and the like. The alkyl alcohol may have one or more substituents, including a halogen atom such as a fluorine atom, a chlorine atom, a bromine atom, an iodine atom and the like. Examples of alkyl alcohols having a halogen atom include halogenated alkyl alcohols having 10 carbon atoms or less, such as chloromethanol, 2-chloroethanol, trifluoromethanol, 2,2,2-trifluoroethanol, perfluoro ethanol, perfluorohexyl alcohol and the like.

In some cases, the alcohol may be an aromatic alcohol, including aryl alcohols having 6 to 20 carbon atoms. Some examples of aryl alcohols include phenol, naphthol and the like. The aryl alcohol may have one or more substituents on the aryl group, including a halogen atom such as a fluorine atom, a chlorine atom, a bromine atom, an iodine atom and the like, or an alkyl group having 20 carbon atoms or less. Examples of aryl alcohols having a halogen atom include halogenated aryl alcohols having 6 to 20 carbon atoms such as pentafluorophenol and the like. Examples of aryl alcohols having an alkyl group include dimethylphenol, trimethylphenol, isopropylphenol, dfisopropylphenol, tert-butylphenol, di-tert-butylphenol and the like.

In some cases, the additive may comprise more than one hydroxyl group. For example, the additive may be a diol or polyol.

In some cases, the additive may be added in an amount that is 0.25 equivalents, 0.5 equivalents, 1.0 equivalent, 1.5 equivalents, 2.0 equivalents, or greater, based on the amount of the imine substrate.

In some cases, the additive may be added as a neat reagent or added as a solution in a solvent. In some cases, the additive may be one or more compounds.

In one set of embodiments, high catalytic activity and enantioselectivity may be observed when water or an alcohol such as n-butanol is used as an additive in the asymmetric cyanation of imines, using the titanium catalysts described herein. In some embodiments, substantially complete conversion of the imine substrate to the desired optically active alpha-aminonitrile can be achieved in 15 minutes with the addition of 1.0 equivalent of n-butanol. In some cases, enantioselectivities of at least 80% ee, at least 85% ee, at least 90% ee, at least 95% ee, at least 98% ee, can be observed. In a particular embodiment, the asymmetric cyanation of imines may be performed with 2.5 to 5 mole % of a titanium catalyst as described herein, at room temperature, to produce a product in >99% yield and having up to 98% ee, in 15 minutes.

In some embodiments, a kit may be provided, containing one or more of the above compositions (e.g., a titanium catalyst). A “kit,” as used herein, typically defines a package or an assembly including one or more of the compositions of the invention, and/or other compositions associated with the invention, for example, as previously described. Each of the compositions of the kit may be provided in liquid form (e.g., in solution), in solid form (e.g., a dried powder), etc. The composition may be provided under ambient or dry and/or inert conditions (e.g., under an inert gas such as nitrogen, argon, helium, etc.). A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of;” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of;” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

The present invention is now more specifically illustrated below with reference to Examples. However, the present invention is not restricted to these Examples.

### Example 1

The following example describes the preparation of titanium compounds (e.g., catalysts) using a first method (herein referred to as “Method 1”). A titanium alkoxide (1.0 equivalents) and an optically active ligand (1.0 equivalents) were combined in a vial in dry toluene. The reaction mixture was stirred for approximately 1 hour. Over a fifteen minute period, water (0.5 equivalents) was added to the solution as a 1-0.5 M solution in tetrahydrofuran. The solution was heated to about 90° C. for approximately 2 hours. Any alcohol present in the solution (e.g., released from the titanium alkoxide complex) was azeotroped and the reaction mixture was dried under high vacuum. The resulting yellow powder was washed with anhydrous pentane and dried to yield the titanium catalyst.

### Example 2

The following example describes the preparation of titanium compounds (e.g., catalysts) using a second method (herein referred to as “Method 2”). A titanium alkoxide (1.0 equivalents) and an optically active ligand (1.0 equivalents) were combined in a vial in dry dichloromethane. The reaction mixture was stirred for approximately 1 hour. Over a fifteen minute period, water (0.5 equivalents) was added directly to the solution or as a 1-0.5 M solution in tetrahydrofuran. The solution was stirred at room temperature for about 2 hours. The reaction mixture was dried. The resulting powder was washed with anhydrous pentane and dried to yield the titanium catalyst.

### Example 3

The following example describes the general analysis of titanium catalyst prepared using Method 1 or Method 2 as describe above in Example 1 or 2, respectively. The isolated titanium catalysts were analyzed by NMR, IR, and mass spectroscopic techniques. All complexes formed showed broad NMR signals which may indicate a mixture of oligomeric or a cluster type of compounds.

### Example 4

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 5

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 6

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 7

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 8

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 9

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 10

A titanium catalyst was synthesized according to Method 2 described in Example 2 using the titanium alkoxide and the optically active ligand shown in Table 1. The isolated catalyst was analyzed according to Example 3 and the mass spectroscopic analysis results are shown in Table 1.

### Example 11

A titanium catalyst was synthesized according to Method 2 described in Example 2 using the titanium alkoxide and the optically active ligand shown in Table 1. The isolated catalyst was analyzed according to Example 3 and the mass spectroscopic analysis results are shown in Table 1.

### Example 12

A titanium catalyst was synthesized according to Method 1 described in Example 1 using the titanium alkoxide and the optically active ligand shown in Table 1, except the titanium alkoxide to water ratio was 1:1. The isolated catalyst was analyzed according to Example 3 and the mass spectroscopic analysis results are shown in Table 1.

### Example 13

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 14

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

### Example 15

A titanium catalyst was synthesized according to Method 1 described in Example 1 using the titanium alkoxide and the optically active ligand shown in Table 1. The isolated catalyst was analyzed according to Example 3 and the mass spectroscopic analysis results are shown in Table 1.

TABLE 1
Mass Analysis of Titanium Catalysts

| | Titanium | | | ESI-TOF, | MALDI- |
|---|---|---|---|---|---|
| Example | alkoxide | Ligand | Method | m/z | TOF, m/z |
| 4 | Ti(Oi-Pr)₄ | 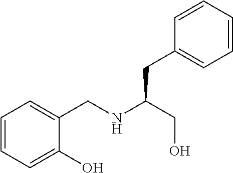 | 1 | Sample is not soluble in most solvents | — |
| 5 | Ti(Oi-Pr)₄ | 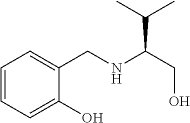 | 1 | 406.94, 463.21, 578.97, 592.95, 748.92, 844.92, 947.40 | 357.35, 429.24, 628.35, 836.34, 1006.49, 1108.41, 1379.49 |
| 6 | Ti(Oi-Pr)₄ | 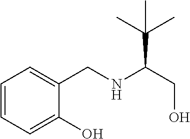 | 1 | 406.94, 578.97, 592.95, 714.05, 736.55, 754.06, 886.61 | 357.21, 485.25, 628.35, 836.34, 1067.44, 1107.42, 1378.50 |
| 7 | Ti(Oi-Pr)₄ | 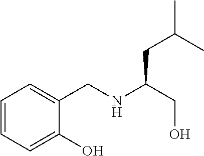 | 1 | 1178.82, 927.66, 886.61, 754.06, 736.54, 721.47, 701.49, 633.41, 475.32 | — |
| 8 | Ti(On-Bu)₄ | 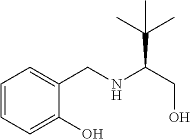 | 1 | 491.24, 631.23, 685.24, 776.32, 790.34, 844.34, 916.32, 981.47, 1003.1, 1075.42 | 494.05, 723.04, 1123.3 |
| 9 | Ti(Ot-Bu)₄ | 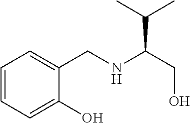 | 1 | 406.94, 493.21, 578.97, 592.95, 844.92, 947.4, 1162.84 | 479.1, 628.23, 732.26, 796.24, 1067.37 |
| 10 | Ti(On-Bu)₄ | 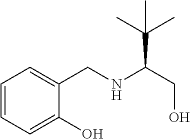 | 2 | 406.94, 578.97, 592.95, 608.94, 754.07, 844.91, 860.91 | — |
| 11 | Ti(On-Bu)₄ | 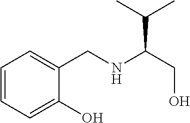 | 2 | 406.94, 463.21, 578.97, 592.95, 735.28, 748.3, 925.41, 947.4 | — |
| 12 | Ti(Ot-Bu)₄ | 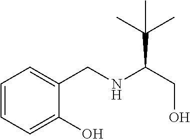 | 1 Ti:H₂O = 1:1 | 592.95, 603.21, 657.22, 694.34, 734.28, 748.30, 802.31, 925.42, 947.40 | — |
| 13 | Ti(Oi-Pr)₄ | 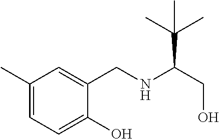 | 1 | 578.96, 592.94, 608.93, 714.05, 768.07 | — |
| 14 | Ti(Oi-Pr)₄ | 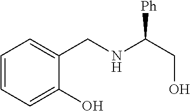 | 1 | 531.17, 553.15, 578.96, 592.94, 671.17, 850.24, 1083.31 | — |
| 15 | Ti(Oi-Pr)₄ | 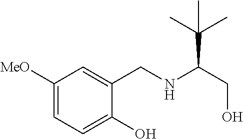 | 1 | 578.96, 592.94, 608.94, 714.05, 784.07 | — |

### Example 16

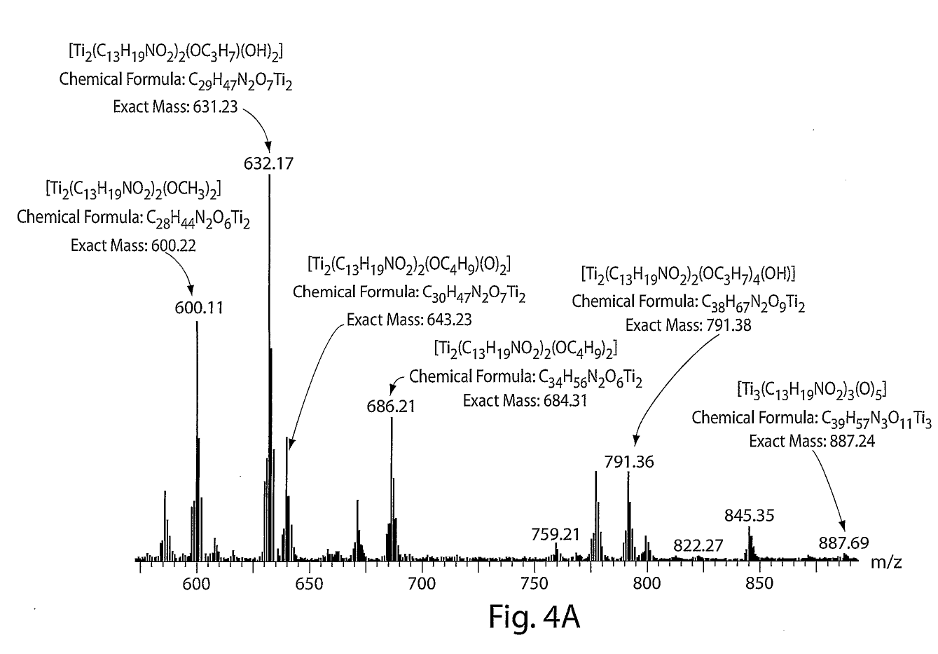

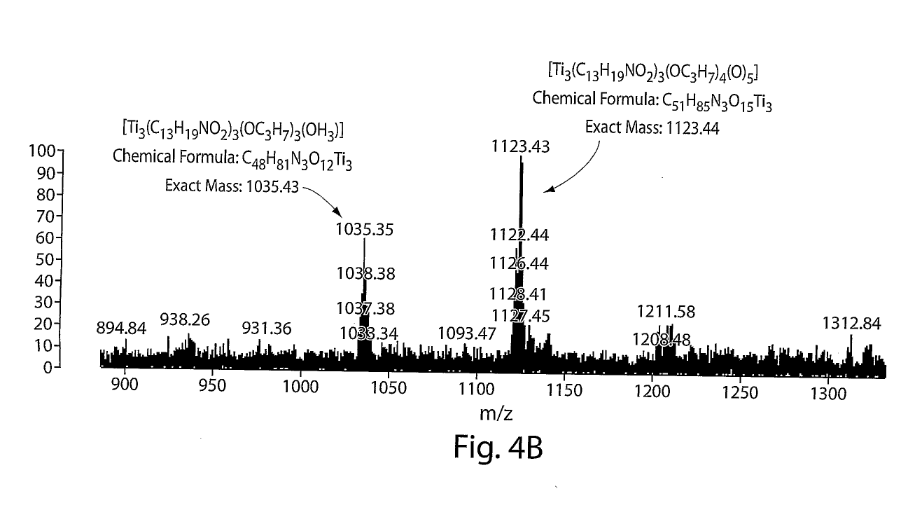

The compounds synthesized in Example 6 and Example 8 may be compared as the same optically active ligand was used. However, although different titanium alkoxides were used, similar mass spectroscopy results were obtained. Without wishing to be bound by theory, this may be due to exchange of the alkoxide groups with methoxide groups from methanol which was used to prepare the mass spectroscopy samples. Table 2 shows the ESI⁺ mass analysis results for the catalysts prepared in Example 6 and Example 8. FIGS. 4A and 4B shows some examples of plausible structural fragments which may correlate to the mass spectroscopy data.

TABLE 2
ESI Mass Analysis Results for Titanium Catalysts Prepared
in Example 6 and Example 8.

| Catalyst Prepare | |
|---|---|
| According to | ESI + Mass Spectroscopy Data |
| Example 6 | 354.98, 386.07, 492.18, 514.13, 586, 600.11, 632.17, |
| | 641, 671, 686.21, 759.21, 777, 800, 845.35, 887.69 |
| Example 8 | 354.98, 386.06, 482.13, 514.13, 586, 600.12, 632.16, |
| | 641, 671.24, 686, 759.3, 777, 786, 800, 871, 925.3, 971 |

### Example 17

The titanium catalysts prepared above were used in the asymmetric cyanation of imines, according to the following procedure. In this example, the catalyst was prepared using Method 1 describe above in Example 1 using the titanium alkoxide and the optically active ligand shown in Table 3. The catalyst (10 mg) was placed in a flask and benzhydryl imine (1 equivalent), trimethylsilyl cyanide (1.5 equivalents relative to the imine substrate) and butanol (1 equivalent relative to the imine) as an additive, were added in this order to a solution comprising the titanium catalyst (FIG. 3A). The reaction was generally carried out in toluene. The resulting material was stirred at room temperature for 1 hour, and NMR and HPLC analysis were carried out to determine the yield and enantiomeric excess (ee) of the product. The results are shown in Table 3

### Example 18

The asymmetric cyanation reaction was carried out in the same manner as in Example 17, except the optically active ligand used is shown in Table 3. The results are shown in Table 3.

### Example 19

The asymmetric cyanation reaction was carried out in the same manner as in Example 18, except 5 mg of the catalyst was used and the reaction was stirred for 2 hours. The results are shown in Table 3.

### Example 20

The asymmetric cyanation reaction was carried out in the same manner as in Example 19, except 2.5 mg of the catalyst was used. The results are shown in Table 3.

### Example 21

The asymmetric cyanation reaction was carried out in the same manner as in Example 20, except the optically active ligand used is shown in Table 3. The results are shown in Table 3.

### Example 22

The asymmetric cyanation reaction was carried out in the same Manner as in Example 20, except the optically active ligand used is shown in Table 3. The results are shown in Table 3.

### Example 23

The asymmetric cyanation reaction was carried out in the same manner as in Example 18, except 1 mg of the catalyst was used. The results are shown in Table 3.

### Example 24

The asymmetric cyanation reaction was carried out in the same manner as in Example 21, except 1 mg of the catalyst was place in a flask. The results are shown in Table 3.

### Example 25

The asymmetric cyanation reaction was carried out in the same manner as in Example 24, except the titanium alkoxide used in shown in Table 3. The results are shown in Table 3.

### Example 26

The asymmetric cyanation reaction was carried out in the same manner as in Example 23, except the titanium alkoxide used in shown in Table 3. The results are shown in Table 3.

### Example 27

The asymmetric cyanation reaction was carried out in the same manner as in Example 25, except the catalyst was prepared using Method 2 as describe in Example 2. The results are shown in Table 3.

### Example 28

The asymmetric cyanation reaction was carried out in the same manner as in Example 26, except the catalyst was prepared using Method 2 as describe in Example 2. The results are shown in Table 3.

TABLE 3
Cyanation of benzhydryl imine using titanium catalysts.

| Titanium Catalyst | | | | Catalyst | | | |
|---|---|---|---|---|---|---|---|
| | Titanium | | Prep. | amount , | Time, | Conv. | |
| Example | alkoxide | Ligand | Method | mg | hr | (%) | ee (%) |
| 17 | Ti(OiPr)₄ | 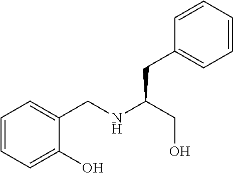 | 1 | 10 | 1 | >99 | 99 |
| 18 | Ti(OiPr)₄ | 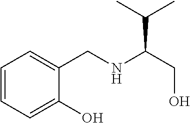 | 1 | 10 | 1 | 98 | 98 |
| 19 | Ti(OiPr)₄ | 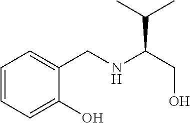 | 1 | 5 | 2 | >99 | 97 |
| 20 | Ti(OiPr)₄ | 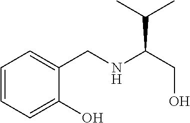 | 1 | 2.5 | 2 | >99 | 97 |
| 21 | Ti(OiPr)₄ | 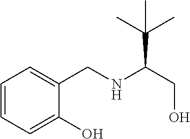 | 1 | 2.5 | 2 | >99 | 96 |
| 22 | Ti(OiPr)₄ | 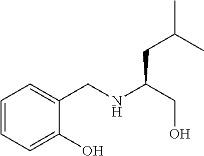 | 1 | 2.5 | 2 | 53 | 93 |
| 23 | Ti(OiPr)₄ | 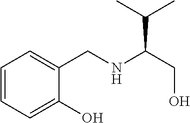 | 1 | 1 | 2 | >99 | 95.6 |
| 24 | Ti(OiPr)₄ | 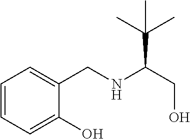 | 1 | 1 | 2 | >99 | 95.8 |
| 25 | Ti(OnBu)₄ | 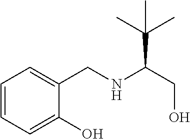 | 1 | 1 | 2 | >99 | 96.1 |
| 26 | Ti(OnBu)₄ | 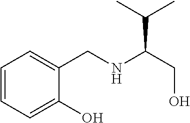 | 1 | 1 | 2 | 90 | 94.1 |
| 27 | Ti(OnBu)₄ | 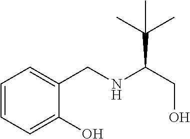 | 2 | 1 | 2 | 97 | 94.3 |
| 28 | Ti(OnBu)₄ | 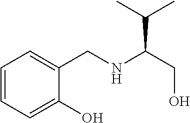 | 2 | 1 | 2 | 97 | 96 |

### Example 29

In this example, a titanium catalyst was prepared using Method 1 describe above in Example 1 using the titanium alkoxide and the optically active ligand shown in Table 4. The resulting titanium catalyst was used in the asymmetric cyanation of benzhydryl imine (FIG. 3A). The catalyst (1 mg) was placed in a flask and benzhydryl imine (1 equivalents), trimethylsilyl cyanide (1.5 equivalents relative to the imine substrate) and butanol (1 equivalent relative to the imine) as an additive, were added in order. The reaction was generally carried out in toluene. The resulting material was stirred at room temperature for 2 hour, and NMR and HPLC analysis were carried out to determine the yield and enantiomeric excess (ee) of the product. The results are shown in Table 4.

### Example 30

The asymmetric cyanation reaction was carried out in the same manner as in Example 29, except the titanium alkoxide to water ratio used in the preparation of the titanium catalyst was 1:1. The results are shown in Table 4.

### Example 31

The asymmetric cyanation reaction was carried out in the same manner as in Example 29, except the titanium alkoxide to water ratio used in the preparation of the titanium catalyst was 1:1.5. The results are shown in Table 4.

### Example 32

The asymmetric cyanation reaction was carried out in the same manner as in Example 29, except the titanium alkoxide used to prepare the catalyst is as given in Table 4. The results are shown in Table 4.

### Example 33

The asymmetric cyanation reaction was carried out in the same manner as in Example 29, except the titanium catalyst was prepared using Method 2 as describe in Example 2. The results are shown in Table 4.

### Example 34

The asymmetric cyanation reaction was carried out in the same manner as in Example 32, except the titanium alkoxide to water ratio used in the preparation of the titanium catalyst was 1:1 and the ratio of the titanium alkoxide to the ligand was 2:1. The results are shown in Table 4.

### Example 35

The asymmetric cyanation reaction was carried out in the same manner as in Example 34, except the ratio of the titanium alkoxide to the ligand was 1:2. The results are shown in Table 4.

### Example 36

The asymmetric cyanation reaction was carried out in the same manner as in Example 29, except the optically active ligand used to prepare the catalyst is as given in Table 4. The results are shown in Table 4.

TABLE 4
Effect of water and ligand ratio during catalyst preparation on the
cyanation of benzhydryl imine.

| | | | | | | Cat. | | | |
|---|---|---|---|---|---|---|---|---|---|
| | Titanium | | | | Prep. | amt, | Time, | Conv. | ee |
| Example | Alkoxide | Ligand | Ti:L | Ti:H₂O | Method | mg | hr | (%) | (%) |
| 29 30 31 32 33 34 35 | Ti(On-Bu)₄ Ti(On-Bu)₄ Ti(On-Bu)₄ Ti(Oi-Pr)₄ Ti(On-Bu)₄ Ti(Oi-Pr)₄ Ti(Oi-Pr)₄ | 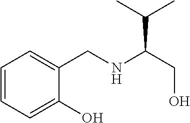 | 1:1 1:1 1:1 1:1 1:1 2:1 1:2 | 1:0.5 1:1.0 1:1.5 1:0.5 1:0.5 1:1.0 1:1.0 | 1 1 1 1 2 1 1 | 1 1 1 1 1 1 1 | 2 2 2 2 2 2 2 | 90 89 85 87 >99 15 99 | 94.1 92.8 91.9 93.4 96 15 91.0 |
| 36 | Ti(On-Bu)₄ | 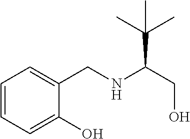 | 1:1 | 1:0.5 | 1 | 1 | 2 | >99 | 96.1 |

### Example 37

In this example, a titanium catalyst was prepared using Method 1 describe above in Example 1 using the titanium alkoxide and the optically active ligand shown in Table 5. The resulting titanium catalyst was used in the asymmetric cyanation of benzyl imine (FIG. 3B) as describe in example 29. NMR and HPLC analysis were carried out to determine the yield and enantiomeric excess (ee) of the product. The results are shown in Table 5.

### Example 38

The asymmetric cyanation reaction was carried out in the same manner as in Example 37, except the titanium catalyst was prepared using Method 2 as describe in Example 2. The results are shown in Table 5.

### Example 39

The asymmetric cyanation reaction was carried out in the same manner as in Example 38, except the titanium alkoxide to water ratio used in the preparation of the titanium catalyst was 1:1. The results are shown in Table 5.

### Example 40

The asymmetric cyanation reaction was carried out in the same manner as in Example 37, except the optically active ligand used to prepare the catalyst is as given in Table 5. The results are shown in Table 5.

### Example 41

The asymmetric cyanation reaction was carried out in the same manner as in Example 40, except the titanium alkoxide to water ratio used in the preparation of the titanium catalyst was 1:1. The results are shown in Table 5.

TABLE 5
Cyanation of Benzyl imine using titanium catalysts.

| | | | | | | Cat. | | | |
|---|---|---|---|---|---|---|---|---|---|
| | Titanium | | | | Prep. | amt, | Time, | Conv. | ee |
| Example | Alkoxide | Ligand | Ti:L | Ti:H₂O | Method | mg | hr | (%) | (%) |
| 37 38 39 | Ti(On-Bu)₄ Ti(On-Bu)₄ Ti(On-Bu)₄ | 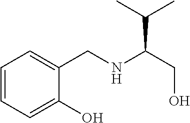 | 1:1 1:1 1:1 | 1:0.5 1:0.5 1:1 | 1 2 2 | 1 1 1 | 2 2 2 | >99 >99 >99 | 72.4 68.8 58.8 |
| 40 41 | Ti(On-Bu)₄ Ti(On-Bu)₄ | 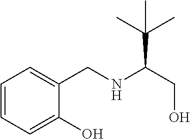 | 1:1 1:1 | 1:0.5 1:1 | 1 2 | 1 1 | 2 2 | >99 >99 | 78.1 76.6 |

### Example 42

In the following example, the asymmetric cyanation reaction was carried out with according to the following general procedure. The optically active titanium catalyst was prepared according to Method 1 described in Example 8. The optically active titanium catalyst was then used directly in the asymmetric cyanation of imines. The optically active titanium catalyst (approximately 1-2 mg) was placed in a flask, and the imine as indicated in Table 6 (0.2 mmol), trimethylsilyl cyanide (1.5 equivalents relative to the imine substrate), and butanol as an additive (1.0 equivalent based on the imine substrate), were added in order. The resulting material was stirred at room temperature for 15-60 min, and NMR and HPLC analysis were carried out to determine the yield and enantiomeric excess (ee) of the product. The results are shown in Table 6.

### Example 43

The asymmetric cyanation reaction was carried out in the same manner as in Example 42, except using the imine substrate as indicated in Table 6. The results are shown in Table 6.

### Example 44

The asymmetric cyanation reaction was carried out in the same manner as in Example 42, except using the imine substrate as indicated in Table 6. The results are shown in Table 6.

### Example 45

The asymmetric cyanation reaction was carried out in the same manner as in Example 42, except using the imine substrate as indicated in Table 6. The results are shown in Table 6.

### Example 46

The asymmetric cyanation reaction was carried out in the same manner as in Example 42, except using the imine substrate as indicated in Table 6. The results are shown in Table 6.

### Example 47

The asymmetric cyanation reaction was carried out in the same manner as in Example 42, except using the imine substrate as indicated in Table 6. The results are shown in Table 6.

### Example 48

The asymmetric cyanation reaction was carried out in the same manner as in Example 42, except using the imine substrate as indicated in Table 6. The results are shown in Table 6.

### Example 49

The asymmetric cyanation reaction was carried out in the same manner as in Example 42, except using the imine substrate as indicated in Table 6. The results are shown in Table 6.

### Example 50

The asymmetric cyanation reaction was carried out in the same manner as in Example 42, except using the imine substrate as indicated in Table 6. The results are shown in Table 6.

### Example 51

The asymmetric cyanation reaction was carried out in the same manner as in Example 42, except using the imine substrate as indicated in Table 6. The results are shown in Table 6.

### Example 52

The asymmetric cyanation reaction was carried out in the same manner as in Example 42, except using the imine substrate as indicated in Table 6. The results are shown in Table 6.

### Example 53

The asymmetric cyanation reaction was carried out in the same manner as in Example 42, except using the imine substrate as indicated in Table 6. The results are shown in Table 6.

### Example 54

The asymmetric cyanation reaction was carried out in the same manner as in Example 42, except using the imine substrate as indicated in Table 6. The results are shown in Table 6.

### Example 55

The asymmetric cyanation reaction was carried out in the same manner as in Example 42, except using the imine substrate as indicated in Table 6. The results are shown in Table 6.

### Example 56

The asymmetric cyanation reaction was carried out in the same manner as in Example 42, except using the imine substrate as indicated in Table 6. The results are shown in Table 6.

### Example 57

The asymmetric cyanation reaction was carried out in the same manner as in Example 42, except using the imine substrate as indicated in Table 6. The results are shown in Table 6.

### Example 58

The asymmetric cyanation reaction was carried out in the same manner as in Example 42, except using the imine substrate as indicated in Table 6. The results are shown in Table 6.

### Example 59

The asymmetric cyanation reaction was carried out in the same manner as in Example 42, except using the imine substrate as indicated in Table 6. The results are shown in Table 6.

### Example 60

The asymmetric cyanation reaction was carried out in the same manner as in Example 42, except using the imine substrate as indicated in Table 6. The results are shown in Table 6.

TABLE 6
Substrate scope for the asymmetric cyanation of imines.

| Example | Imine | Product | Conversion, % | ee, % |
|---|---|---|---|---|
| 42 | 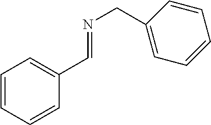 | 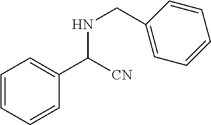 | >99 | 82 |
| 43 | 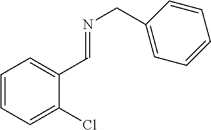 | 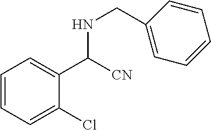 | >99 | 89 |
| 44 | 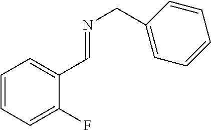 | 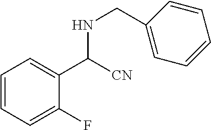 | >99 | 83 |
| 45 | 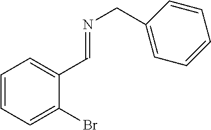 | 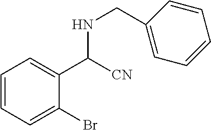 | >99 | 88 |
| 46 | 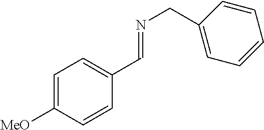 | 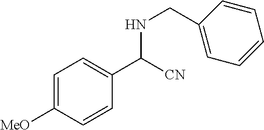 | >99 | 84 |
| 47 | 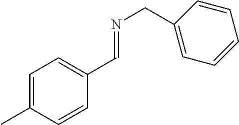 | 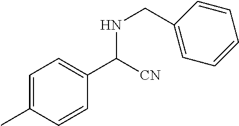 | >99 | 86 |
| 48 | 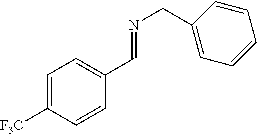 | 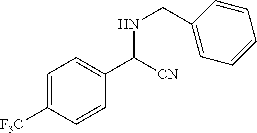 | >99 | 86 |
| 49 | 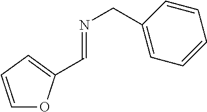 | 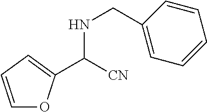 | >99 | 49 |
| 50 | 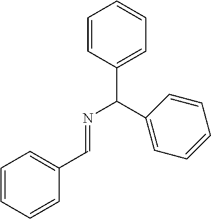 | 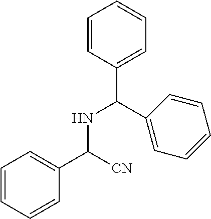 | >99 | 96 |
| 51 | 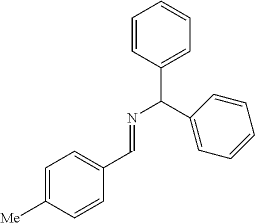 | 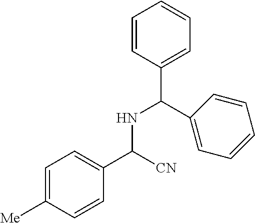 | >99 | 95 |
| 52 | 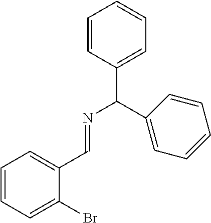 | 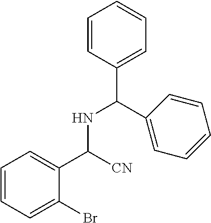 | >99 | 97 |
| 53 | 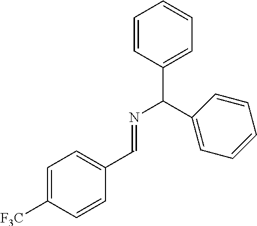 | 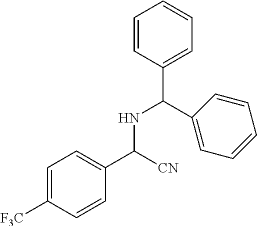 | >99 | 83 |
| 54 | 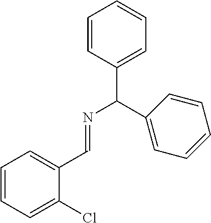 | 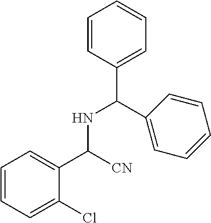 | >97 | 98 |
| 55 | 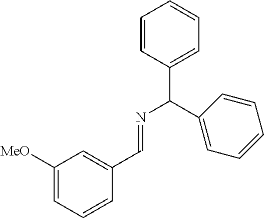 | 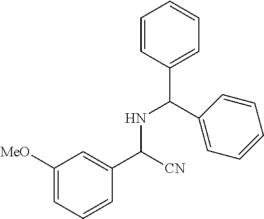 | >99 | 92 |
| 56 | 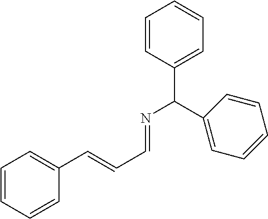 | 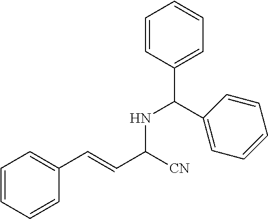 | >99 | 92 |
| 57 | 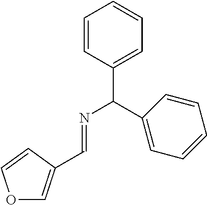 | 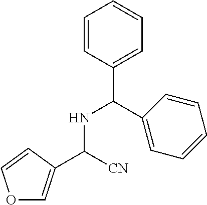 | >97 | 88 |
| 58 | 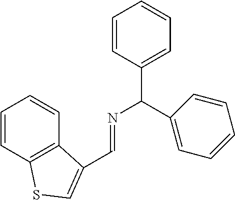 | 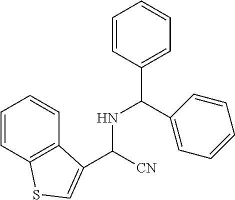 | >99 | 98 |
| 59 | 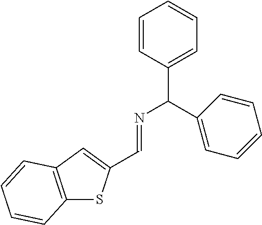 | 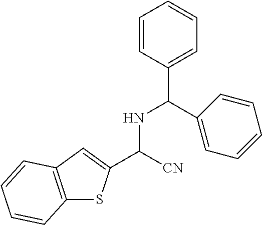 | >99 | 91 |
| 60 | 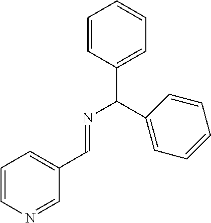 | 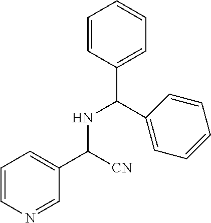 | >99 | 93 |

### Example 60

A catalyst was prepared according to example 4 was used in an asymmetric cyanation reaction according to the following procedure. The catalyst (5 mg) was dispersed in toluene, vortexed to produce a fine suspension, centrifuged, and the soluble part was separated by decanting the liquid. The solid component was again washed two times with fresh toluene.

The catalyst was charged with 700 μL of toluene, 0.2 mmol benzhydryl imine, 1.5 equiv. TMSCN, 1.0 equiv n-BuOH, and the reaction was carried out at room temperature for 2 hours (FIG. 3A). After the 2 hours, the reaction mixture was centrifuged to separate the solid catalyst from the reaction mixture. The liquid components were passed through celite and washed with dichloromethane. The volatile components were evaporated and the product was analyzed by HPLC to determine the enantiomeric excess (ee) of the product and was analyzed by NMR to determine the percent conversion. The results are shown in Table 7.

The isolated solid catalyst was recycled and used again for the cyanation of benzhydryl imine as described in Example 61.

### Example 61

The isolated solid catalyst isolated from example 60 was charged with fresh reactants and solvent as described in Example 60. The results are shown in Table 7. The isolated solid catalyst was used again for the cyanation of benzhydryl imine as described in Example 62.

### Example 62

The separated solid catalyst isolated from example 61 was charged with fresh reactants and solvent as described in Example 60. The results are shown in Table 7. The isolated solid catalyst was recycled and used again for the cyanation of benzhydryl imine as described in Example 63.

### Example 63

The separated solid catalyst isolated from example 62 was charged with fresh reactants and solvent as described in Example 60. The results are shown in Table 7. The isolated solid catalyst was recycled and used again for the cyanation of benzhydryl imine as described in Example 64.

### Example 64

The separated solid catalyst isolated from example 63 was charged with fresh reactants and solvent as described in Example 60. The results are shown in Table 7. The isolated solid catalyst was recycled and used again for the cyanation of benzhydryl imine as described in Example 65.

### Example 65

The separated solid catalyst isolated from example 64 was charged with fresh reactants and solvent as described in Example 60. The results are shown in Table 7. The isolated solid catalyst was recycled and used again for the cyanation of benzhydryl imine as described in Example 63.

### Example 66

The separated solid catalyst isolated from example 65 was charged with fresh reactants and solvent as described in Example 60. The results are shown in Table 7. The isolated solid catalyst was recycled and used again for the cyanation of benzhydryl imine as described in Example 67.

### Example 67

The separated solid catalyst isolated from example 66 was charged with fresh reactants and solvent as described in Example 60. The results are shown in Table 7. The isolated solid catalyst was recycled and used again for the cyanation of benzhydryl imine as described in Example 68.

### Example 68

The separated solid catalyst isolated from example 67 was charged with fresh reactants and solvent as described in Example 60. The results are shown in Table 7. The isolated solid catalyst was recycled and used again for the cyanation of benzhydryl imine as described in Example 69.

### Example 69

The separated solid catalyst isolated from example 68 was charged with fresh reactants and solvent as described in Example 60. The results are shown in Table 7. The isolated solid catalyst was recycled and used again for the cyanation of benzhydryl imine as described in Example 70.

### Example 70

The separated solid catalyst isolated from example 69 was charged with fresh reactants and solvent as described in Example 60. The results are shown in Table 7.

### Example 71

An asymmetric cyanation reaction was conducted according to Example 60 using a catalyst prepared according to example 5. The results are shown in Table 7. The isolated solid catalyst was recycles and used again for the cyanation of benzhydryl imine as described in Example 72.

### Example 72

The separated solid catalyst isolated from example 71 was charged with fresh reactants and solvent as described in Example 60. The results are shown in Table 7. The isolated solid catalyst was recycled and used again for the cyanation of benzhydryl imine as described in Example 73.

### Example 73

The separated solid catalyst isolated from example 72 was charged with fresh reactants and solvent as described in Example 60. The results are shown in Table 7. The isolated solid catalyst was recycled and used again for the cyanation of benzhydryl imine as described in Example 74.

### Example 74

The separated solid catalyst isolated from example 73 was charged with fresh reactants and solvent as described in Example 60. The results are shown in Table 7.

TABLE 7
Cyanation of benzhydryl imine using recycled titanium catalysts.

| Example | Conv. % | ee, % |
|---|---|---|
| 60 | >99.9 | 97.3 |
| 61 | >99.9 | 96.9 |
| 62 | >99.9 | 97.0 |
| 63 | >99 | 96.8 |
| 64 | >99 | 96.9 |
| 65 | >99 | 96.0 |
| 66 | >99 | 96.3 |
| 67 | >99 | 96.3 |
| 68 | >99 | 96 |
| 69 | >99 | 95.9 |
| 70 | >99 | 94.6 |
| 71 | >99.9 | 95.3 |
| 72 | >99.9 | 96.3 |
| 73 | >99.9 | 96.5 |
| 74 | >99 | 96.2 |

### Example 75

The following example describes the preparation of a titanium compound (e.g., catalysts) using a third method (herein referred to as “Method 3”). A titanium alkoxide,
(Ti(Oi-Pr)₄) (1.0 equivalents), and an optically active ligand (1.0 equivalents) were combined in a vial in dry toluene. The reaction mixture was stirred for approximately 1 hour. Over a fifteen minute period, water (0.5 equivalents) was added to the solution as a 1-0.5 M solution in tetrahydrofuran. The solution was heated to about 90° C. for approximately 2 hours followed by stirring under reflux for 1 hour. The reaction mixture was cooled to room temperature and the precipitate was filtered, washed with toluene, and dried under vacuum. The catalyst was isolated as a yellow powder.

### Example 76

A titanium catalyst was synthesized according to Method 3 described in Example 75 using the titanium alkoxide and the optically active ligand shown in Table 1 for Example 4.

The isolated catalyst was used for the asymmetric cyanation according to the procedure described in Example 60. The product was analyzed by HPLC to determine the enantiomeric excess (ee) of the product and was analyzed by NMR to determine the percent conversion. The results are shown in Table 8. The catalyst isolated from the reaction mixture was recycled and used again in the cyanation of benzhydryl amine as described in Example 77.

### Example 77

The isolated solid catalyst isolated from example 76 was charged with fresh reactants and solvent as described in Example 60. The results are shown in Table 8. The isolated solid catalyst was recycled and used again for the cyanation of benzhydryl imine described in Example 78.

### Example 78

The isolated solid catalyst isolated from example 77 was charged with fresh reactants and solvent as described in Example 60. The results are shown in Table 8. The isolated solid catalyst was recycled and used again for the cyanation of benzhydryl imine as described in Example 79.

### Example 79

The isolated solid catalyst isolated from example 78 was charged with fresh reactants and solvent as described in Example 60. The results are shown in Table 8. The isolated solid catalyst was recycled and used again for the cyanation of benzhydryl imine as described in Example 80.

### Example 80

The isolated solid catalyst isolated from example 79 was charged with fresh reactants and solvent as described in Example 60. The results are shown in Table 8. The isolated solid catalyst was recycled and used again for the cyanation of benzhydryl imine as described in Example 81.

### Example 81

The isolated solid catalyst isolated from example 79 was charged with fresh reactants and solvent as described in Example 60. The results are shown in Table 8.

TABLE 8
Cyanation of benzhydryl imine using recycled titanium catalysts.

| Example | Conv. % | ee, % |
|---|---|---|
| 76 | >99.9 | 96.7 |
| 77 | >99.9 | 96.2 |
| 78 | >99.9 | 96.3 |
| 79 | >99.9 | 95.5 |
| 80 | >99.9 | 95.4 |
| 81 | >99.9 | 94.2 |

### Example 82

A titanium catalyst was synthesized according to Method 3 described in Example 75 using the titanium alkoxide and the optically active ligand shown in Table 9. The isolated catalyst was used for the asymmetric cyanation according to the procedure described in Example 60 and the results are shown in Table 9.

### Example 83

A titanium catalyst was synthesized according to Method 3 described in Example 75 using the titanium alkoxide and the optically active ligand shown in Table 9. The isolated catalyst was used for the asymmetric cyanation according to the procedure described in Example 60 and the results are shown in Table 9.

TABLE 9
Cyanation of benzhydryl imine using titanium catalysts.

| Ex- | | Conv. | ee, |
|---|---|---|---|
| ample | Ligand | % | % |
| 82 | 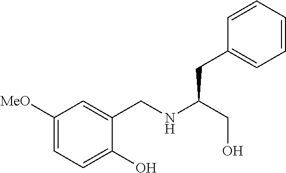 | >99 | 97.2 |
| 83 | 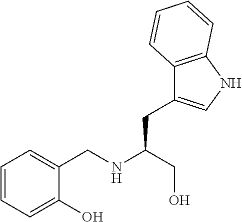 | 96 | 94.7 |

### Example 84

The following examples describe methods to determine whether an isolated solid material is an active catalyst and/or whether a component in solution is an active catalyst. The solid catalyst isolated in Example 75 was used for the asymmetric cyanation according to the procedure described in Example 60. The solid catalyst was separated from the reaction mixture by centrifugation. A small aliquot of the solution from the catalyst formation reaction was analyzed by HPLC and NMR. The results are shown in Table 10. The remaining solution was subjected to further cyanation reactions as described in Example 85.

### Example 85

To the remaining solution from Example 84 was added benzhydryl imine (0.2 mmol), TMSCN (1.5 equiv), butanol (1.0 equiv), and toluene (500 μL). After 2 hours, the solvents were evaporated and the product was analyzed by HPLC and NMR to determine the enantiomeric excess (ee) and percent conversion of the product, respectively. The results are given in Table 10.

### Example 86

To the solid catalyst isolated in Example 75 was added benzhydryl imine (0.2 mmol), butanol (1.0 equiv), and toluene (500 μL). After 2 hours, the solid catalyst was separated from the reaction mixture by centrifugation. To the liquid components of the reaction mixture was added TMSCN (1.5 equiv) and a cyanation reaction was carried out as described in Example 60. The results are given in Table 10.

TABLE 10
Cyanation of benzhydryl imine.

| Example | Conv. % | ee, % |
|---|---|---|
| 84 | >99.9 | 96.7 |
| 85 | 25 | 18.1 |
| 86 | 27 | 7.5 |

### Example 87

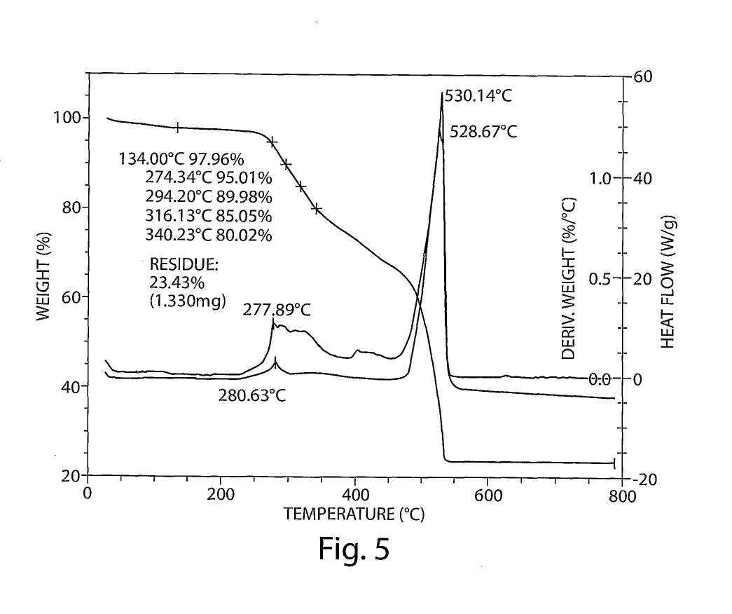

The solid catalyst isolated in Example 4 was analyzed by Thermogravimetric analysis under air (FIG. 5) to determine titanium content. The titanium content was found to be 14% which closely corresponds to a molecular formula of Ti₃(C₁₆H₁₇NO₂)₃(OH)₆ in which titanium content is 14.20% or Ti₃(C₁₆H₁₇NO₂)₃(O)₃(OH)₃ in which titanium content is 14.245%.

### Example 88

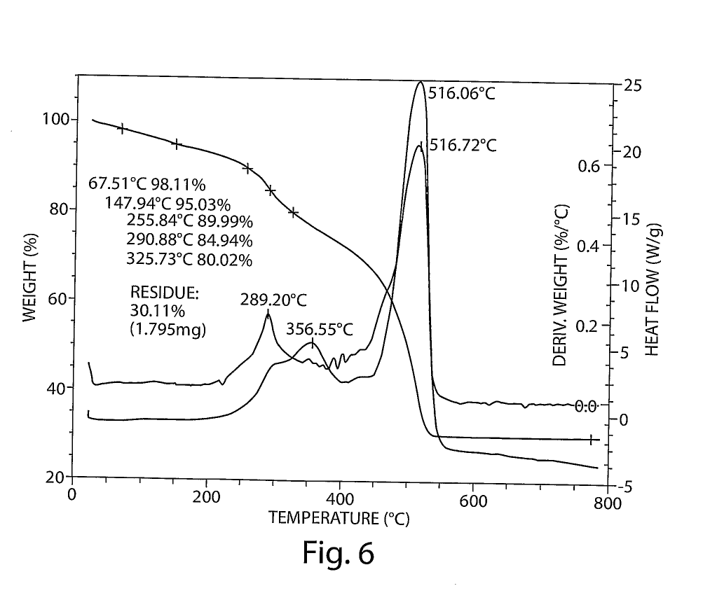

The solid catalyst isolated in Example 5 was analyzed by Thermogravimetric analysis under air (FIG. 6) to determine titanium content. The titanium content was found to be 18% which closely corresponds to a molecular formula of Ti₃(C₁₂H₁₉NO₂)₂(C₃H₇O)₃(OH)₃ in which titanium content is 18.26%

### Example 89

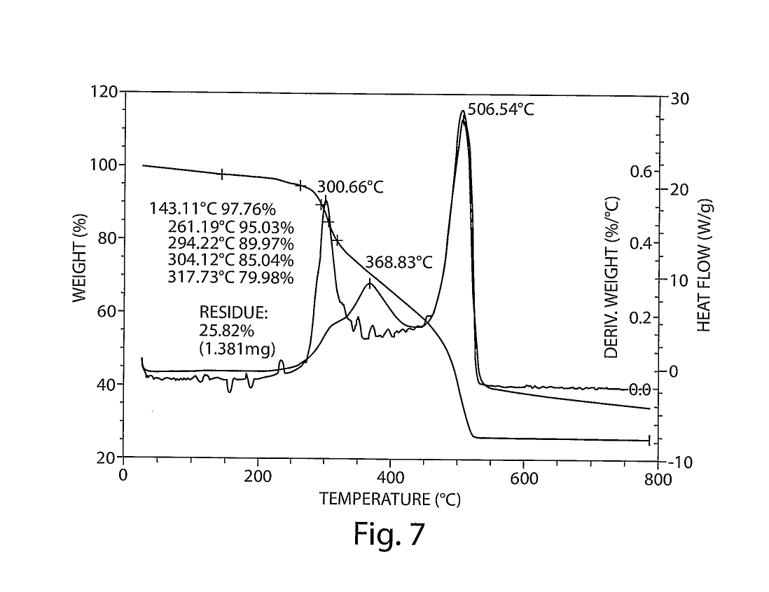

The solid catalyst isolated in Example 6 was analyzed by Thermogravimetric analysis under air (FIG. 7) to determine titanium content. The titanium content was found to be 15.5% which closely corresponds to a molecular formula of Ti₃(C₁₃H₁₉NO₂)₃(OH)₆ in which titanium content is 15.79% or Ti₃(C₁₃H₂₁NO₂)₃(O)₃(OH)₃ in which titanium content is 15.84%.

### Example 90

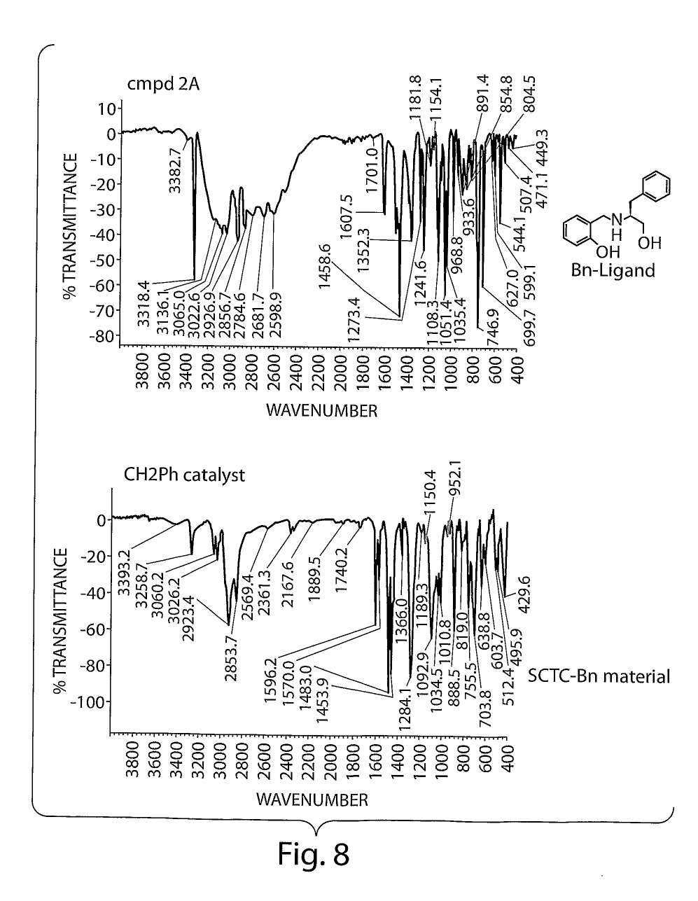

The solid catalyst isolated in Example 4 was analyzed by IR spectroscopy in the range of 4000-400 cm⁻¹ and compared with the free ligand (FIG. 8). The free OH groups in the ligands disappeared upon formation of the catalyst. The shift in NH vibration to 3258 cm⁻¹ compared to 3318 cm⁻¹ of the free ligand indicated that nitrogen also coordinated to the Ti. The lack of a peak at about 940 cm⁻¹ indicated the absence of terminal Ti═O bonds. Peaks at 703 cm⁻¹ suggest the presence of Ti—O—Ti structure.

### Example 91

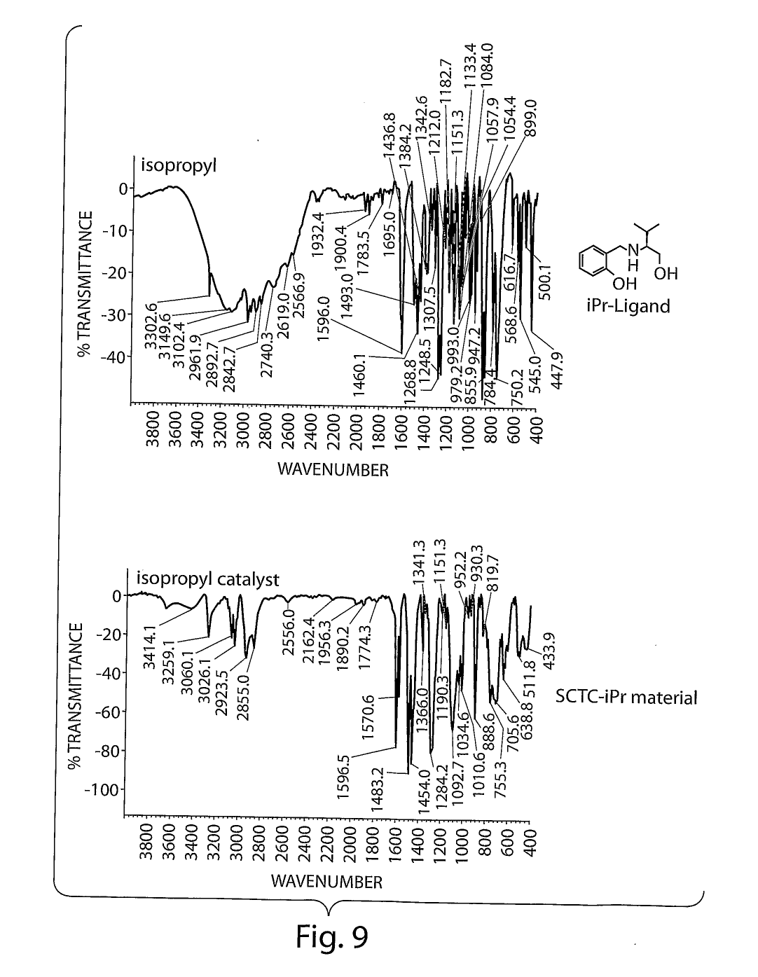

The solid catalyst isolated in Example 5 was analyzed by IR spectroscopy in the range of 4000-400 cm⁻¹ (FIG. 9) and was compared with the free ligand. The free OH groups in the ligands disappeared upon formation of the catalyst. The shift in NH vibration to 3259 cm⁻¹ compared to 3302 cm⁻¹ of the free ligand indicated that nitrogen also coordinated to the Ti. The lack of a peak at about 940 cm⁻¹ indicated the absence of terminal Ti═O bonds. Peaks at 705 cm⁻¹ suggest the presence of Ti—O—Ti structure.

### Example 92

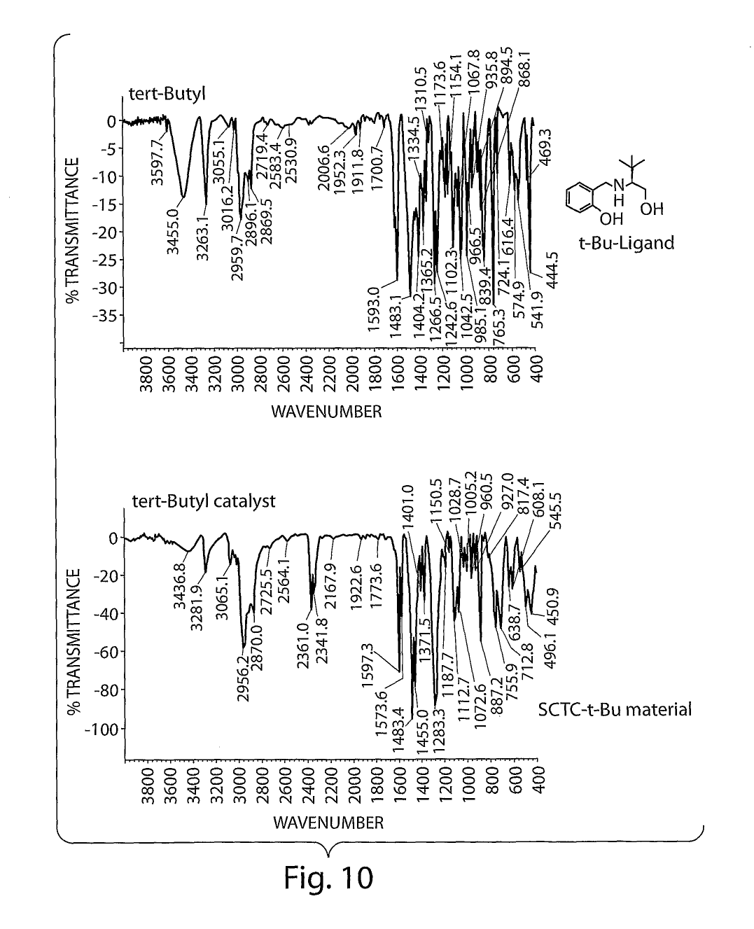

The solid catalyst isolated in Example 6 was analyzed by IR spectroscopy in the range of 4000-400 cm⁻¹ and compared with the free ligand (FIG. 10). The free OH groups in the ligands were disappeared upon formation of the catalyst. The shift in NH vibration to 3281 cm⁻¹ compared to 3263 cm⁻¹ of the free ligand indicated that nitrogen also coordinated to the Ti. Lack of peak at about 940 cm⁻¹ show the absence of terminal Ti═O bonds. Peaks at 712 cm⁻¹ suggests the presence of Ti—O—Ti structure.

### Example 93

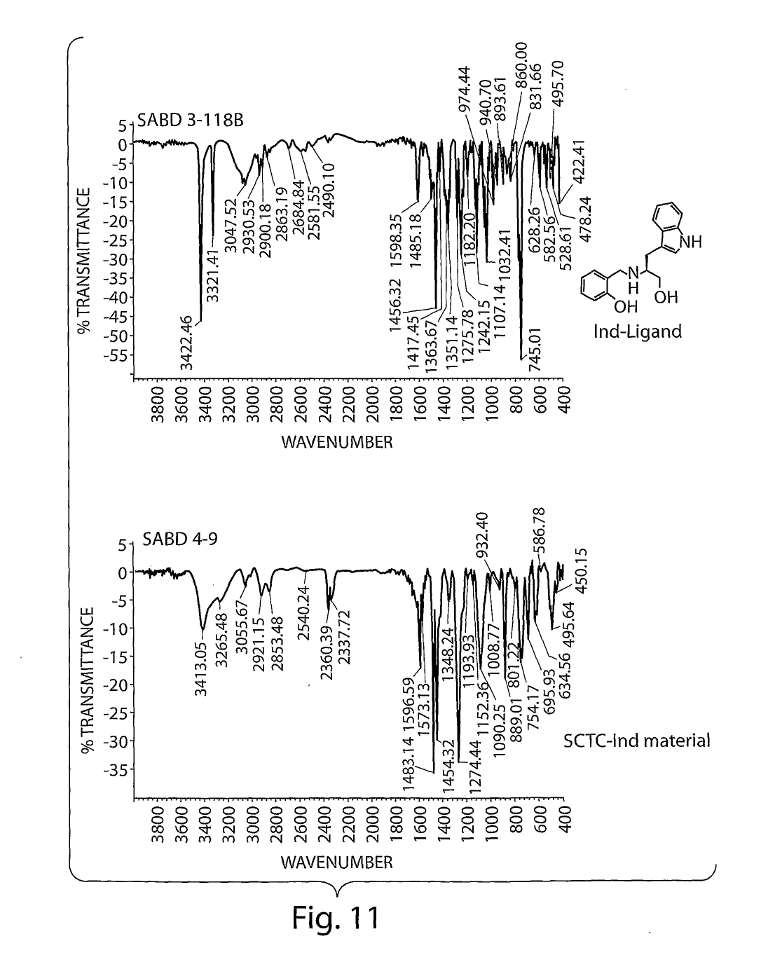

The solid catalyst isolated in Example 85 was analyzed by IR spectroscopy in the range of 4000-400 cm⁻¹ (FIG. 11)and compared with the free ligand. The free OH groups in the ligands disappeared upon formation of the catalyst. The shift in NH vibrations to 3413 cm⁻¹ and 3265 cm⁻¹ compared to 3422 cm⁻¹ and 3321 cm⁻¹ of the free ligand indicated that nitrogen also coordinated to the Ti. The lack of a peak at about 940 cm⁻¹ indicated the absence of terminal Ti═O bonds. Peaks at 695 cm⁻¹ suggest the presence of Ti—O—Ti structure.

### Example 94

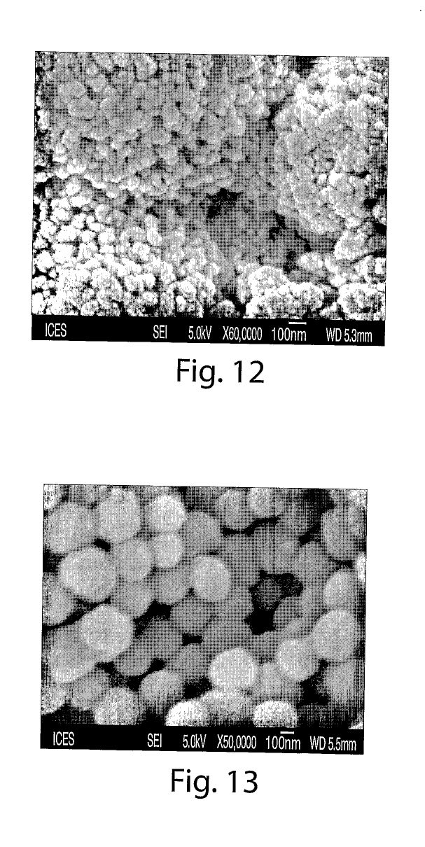

The solid catalyst isolated in Example 4 was analyzed by Scanning Electron Microscopy (SEM) analysis. The SEM image shown in FIG. 12 indicates that the particles are aggregates of irregular nanospheres of about 10-50 nm in size.

### Example 95

The solid catalyst isolated in Example 83 was analyzed by Scanning Electron Microscopy (SEM) analysis. The SEM image shown in FIG. 13 indicates that the material has a regular microspherical structure of 300 nm in size.

### Example 96

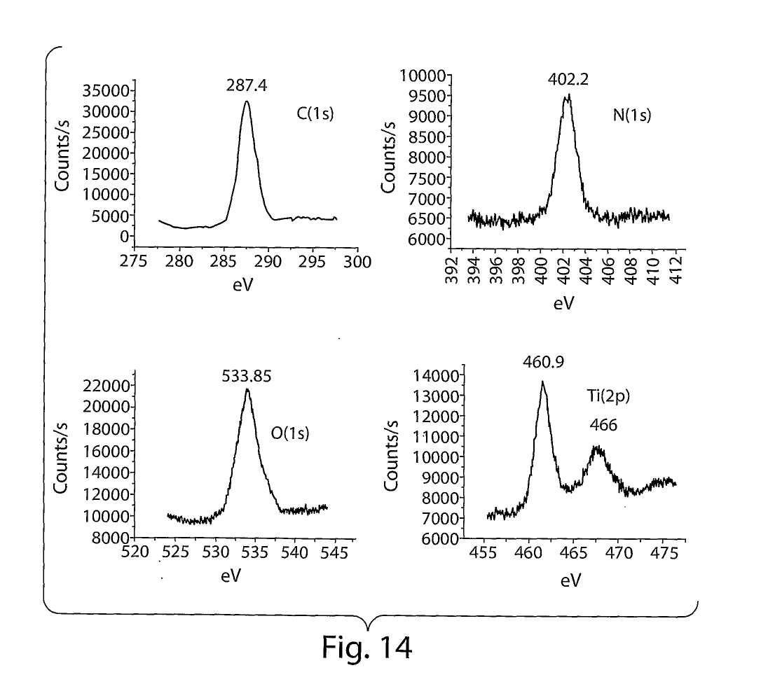

The solid catalyst isolated in Example 4 was analyzed by X-ray Photoelectron Spectroscopy (XPS), as shown in FIG. 14. Peaks observed were C(1s), 287.4 eV; N(1s), 402.2 eV; 0(1s), 533.85 eV and Ti(2p), 460.9 eV & 466 eV.

### Comparative Example 1

A chiral titanium catalyst was prepared from 1 mol % of chiral aminoalcohol ligand (0.5 mg of t-Bu ligand, N-(2′-Hydroxy-3′-ethoxy-phenyl)methyl-(S)-2-amino-3,3-dimethyl-butanol, in 0.3 mL of toluene) and 1 mol % of partially hydrolyzed Ti(OnBu)₄ (0.05 mL of 0.05M toluene solution of PHTA, conventional catalyst) obtained by treating Ti(OnBu)₄ monomer in presence of 0.11 to 0.15 equivalents of water in toluene (0.65 mL, 190 ppm of moisture). To this chiral catalyst solution. N-benzylidene-1-phenylmethanamine (0.2 mmol) was added, followed by 1.5 equivalents of TMSCN and 1 equivalent of n-BuOH. After one hour of reaction, a 50% yield and 42% ee was observed. The catalyst was not separated from the reaction mixture and hence was not recycled.

What is claimed:

1. A titanium catalyst, produced by bringing water into contact with a complex prepared from a titanium alkoxide represented by the general formula (d),
Ti(OR′)ₓY₍₄₋ₓ₎ (d),
wherein
R′ can be the same or different and is an alkyl, an alkenyl, or an aryl group, optionally substituted;
Y can be the same or different and in a halogen atom, an acyl group, or an acetylacetonate group; and
x is an integer having a value between 0 and 4; and
a ligand represented by the formula (e), 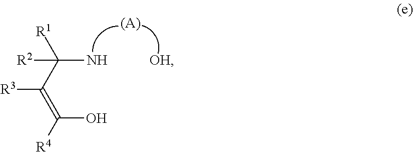
wherein
R¹, R², R³, and R⁴ are independently a hydrogen atom, an alkyl group, an alkenyl group, an aryl group, an aromatic heterocyclic group, a non-aromatic heterocyclic group, an acyl group, a hydroxyl group, an alkoxy group, an aryloxy group, an amino group, a cyano group, a nitro group, a silyl group, a siloxy group, an alkoxycarbonyl group or an aryloxycarbonyl group, each of which may have a substituent, or two or more of R¹, R², R³, and R⁴ may be linked together to form a ring, and the ring may have a substituent; and
(A) represents a group with two or more carbon atoms.

2. An isolated titanium catalyst for synthesis reactions comprising a compound represented by the general formula (f),
[Tiₘ(L)ₙ(OR′)ₚ(O)_{q}(OH)ᵣYₛ] (f)
wherein
R′ can be the same or different and is an alkyl, an alkenyl, or an aryl group, optionally substituted;
Y can be the same or different and is a halogen atom, an acyl group, or an acetylacetonate group;
m is an integer greater than 1;
n and q are the same or different and are 0 or integers greater than 0;
p, r, and s are the same or different and are 0 or an integer greater than 0; and
L is a ligand represented by the general formula (e), 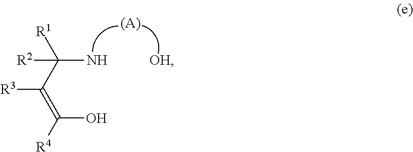
wherein
R¹, R², R³, and R⁴ are independently a hydrogen atom, an alkyl group, an alkenyl group, an aryl group, an aromatic heterocyclic group, a non-aromatic heterocyclic group, an acyl group, a hydroxyl group, an alkoxy group, an aryloxy group, an amino group, a cyano group, a nitro group, a silyl group, a siloxy group, an alkoxycarbonyl group or an aryloxycarbonyl group, each of which may have a substituent, or two or more of R¹, R², R³, and R⁴ may be linked together to form a ring, and the ring may have a substituent; and
(A) represents a group with two or more carbon atoms.

3. An isolated titanium catalyst as claim 2, wherein the compound is represented by any one of the following formulas:
[Tiₘ(L)ₙ(OH)ᵣ] (g),
[Tiₘ(L)ₙ(O)_{q}(OH)ᵣ] (h), or
[Tiₘ(L)ₙ(OR′)ₚ(OH)ᵣ] (i).

4. An isolated titanium catalyst as claim 3, wherein the compound is represented by any one of the following formulas:
[Ti₃(L)₃(OH)₆] (j),
[Ti₃(L)₃(O)₃(OH)₃] (k), or
[Ti₃(L)₂(OR′)₃(OH)₃] (l).

5. A titanium catalyst as in any preceding claim, wherein, when coordinated to titanium, L is a monovalent ion, a divalent ion, or a trivalent ion.

6. A titanium catalyst as in any preceding claim, wherein, when coordinated to titanium, L is a divalent ion.

7. A titanium catalyst as in any preceding claim, wherein (A) represents a group with two or more carbon atoms having an asymmetric carbon atom or axial asymmetry.

8. A titanium catalyst as in any proceeding claim, wherein the titanium complex is isolated as a solid.

9. A process for the preparation of a titanium catalyst according to any preceding claim, comprising the steps of
a) forming a solution comprising of a complex prepared from a titanium alkoxide represented by the general formula (d) and a ligand represented by the general formula (e);
b) contacting water with the solution to give solution or suspension of the catalyst; and
c) removing the solvent from the solution or suspension of the catalyst.

10. A titanium catalyst as in any preceding claim, wherein the ligand represented by said general formula (e) is represented by general formula (a), 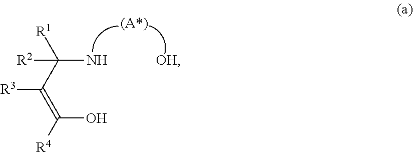
wherein
R¹, R², R³, and R⁴ are independently a hydrogen atom, an alkyl group, an alkenyl group, an aryl group, an aromatic heterocyclic group, a non-aromatic heterocyclic group, an acyl group, an alkoxycarbonyl group or an aryloxycarbonyl group, each of which may have a substituent, or two or more of R¹, R², R³, and R⁴ may be linked together to form a ring, and the ring may have a substituent; and
(A*) represents a group with two or more carbon atoms having an asymmetric carbon atom or axial asymmetry.

11. A titanium catalyst as in any preceding claim, wherein the ligand represented by said general formula (e) is represented by general formula (b), 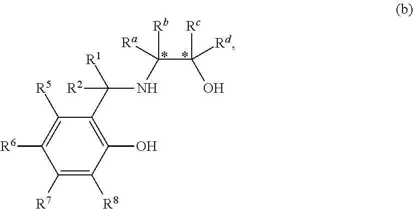
wherein
R^{a}, R^{b}, R^{c}, and R^{d} are each a hydrogen atom, an alkyl group, an aryl group, alkoxycarbonyl group, an aryloxycarbonyl group or an aminocarbonyl group, each of which may have a substituent, or two or more of R^{a}, R^{b}, R^{c}, and R^{d} may be linked together to form a ring, and the ring may have a substituent; at least one of R^{a}, R^{b}, R^{c}, and R^{d} is a different group; both or at least one of the carbon atoms indicated as * become an asymmetric center; and parts indicated as (NH) and (OH) do not belong to (A*), and represent an amino group and a hydroxyl group, respectively, corresponding to those in said general formula (a) to which (A*) is bonded; and
R⁵, R⁶, R⁷, and R⁸ are independently a hydrogen atom, a halogen atom, an alkyl group, an alkenyl group, an aryl group, an aromatic heterocyclic group, a non-aromatic heterocyclic group, an alkoxycarbonyl group, an aryloxycarbonyl group, a hydroxyl group, an alkoxy group, an aryloxy group, an amino group, a cyano group, a nitro group, a silyl group or a siloxy group which may have a substituent, each of which may be linked together to form a ring.

12. A titanium catalyst as in claim 11, wherein R^{a} is methyl, ethyl, n-propyl, iso-propyl, n-butyl, iso-butyl, sec-butyl, tert-butyl, or benzyl, and R^{b}, R^{e}, and R^{d} are hydrogen atoms.

13. A titanium catalyst as in any preceding claim, wherein the ligand has the structure,

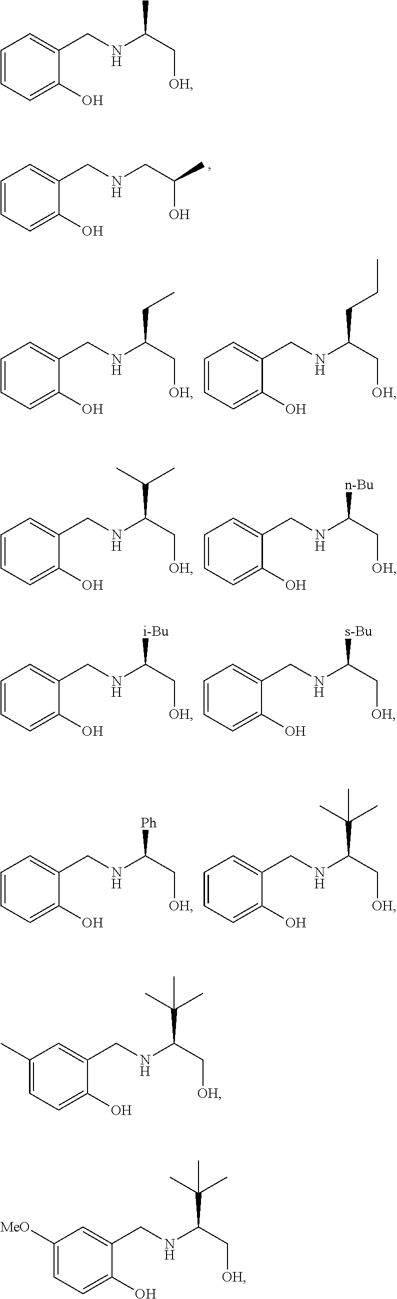

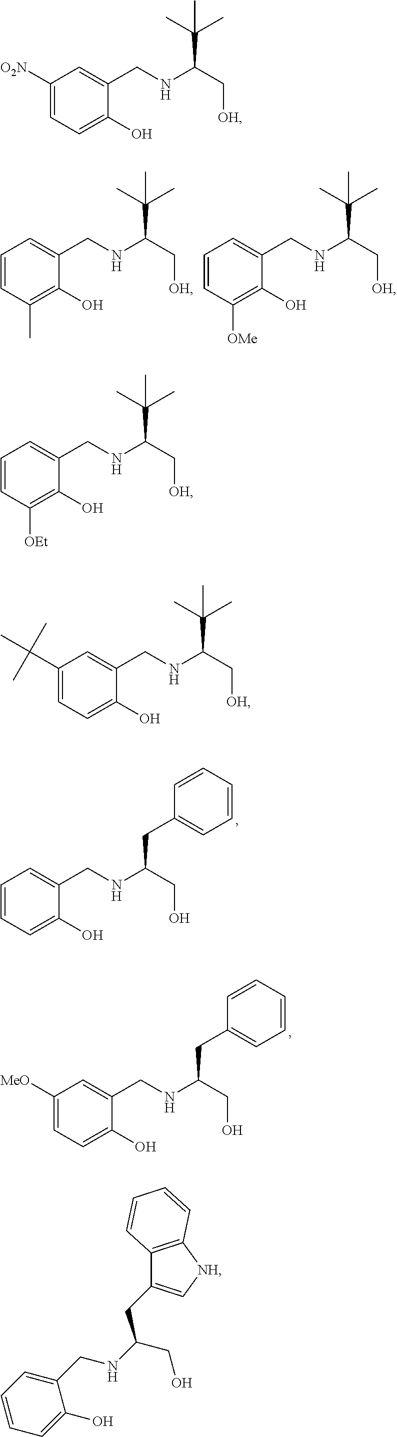

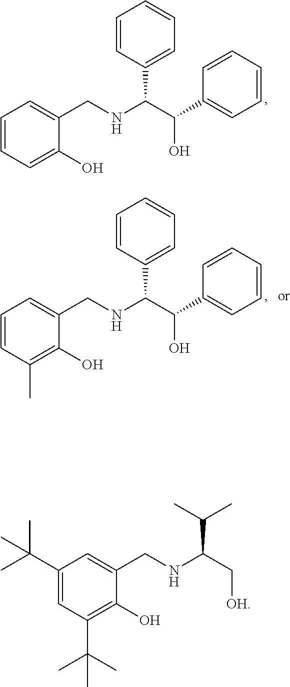

14. A process for cyanation of inclines, comprising reacting an imine with a cyanating agent in the presence of the titanium catalyst of any preceding claim.

15. A process for cyanation of imines, wherein the process is an asymmetric reaction in the presence of a titanium catalyst of any one of claims 2-8 and 10-13.

16. A process for cyanation of imines as in any preceding claim, wherein the process is carried out in presence of an additive having at least one hydroxyl group.

17. A process for cyanation of imines as in any preceding claim, wherein the imine is represented by the general formula (c), 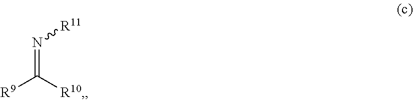
wherein
R⁹ and R¹⁰ are independently a hydrogen atom, an alkyl group, an alkenyl group, an alkynyl group, an aryl group, an aromatic heterocyclic group or a non-aromatic heterocyclic group, each of which may have a substituent, and R⁹ is different from R¹⁰;
R⁹ and R¹⁰ may be linked together to form a ring, and the ring may have a substituent;
R¹¹ is a hydrogen atom, an alkyl group, an alkenyl group, an alkynyl group, an aryl group, an aromatic heterocyclic group or a non-aromatic heterocyclic group, a phosphonate, phosphinoyl, phosphine oxide, alkoxycarbonyl, sulfinyl, or sulfoxy group, each of which may have a substituent; and
R¹¹ may be linked either to R⁹ or R¹⁰ to form a ring through a carbon chain, and the ring may have substituents.

18. A process for cyanation of imines as in any preceding claim, wherein the cyanating agent is hydrogen cyanide, trialkylsilyl cyanide, acetone cyanohydrin, cyanoformate ester, potassium cyanide-acetic acid, potassium cyanide-acetic anhydride, or tributyltin cyanide.

19. A process for cyanation of imines as in any preceding claim, wherein the cyanating agent is trialkylsilyl cyanide.

20. A process for cyanation of imines as in any preceding claim, wherein the cyanating agent is mixture of trialkylsilyl cyanide and hydrogen cyanide.

21. A process for cyanation of imines as in claim 15, wherein the additive is an alcohol, diol, polyol, phenol, or water.

22. A kit comprising,
a composition of any preceding claim.
